SEQUENCE LISTING (60 sequences)

SEQ ID NO: 1 - DNA, 12 bp, Artificial sequence
  vector
cagccctgcagg

SEQ ID NO: 2 - DNA, 34 bp, Artificial sequence
  vector
taactataacggtcctaaggtagcgacactagtg

SEQ ID NO: 3 - protein, 6 aa, Artificial sequence
  synthetic peptide motif
KVATLV

SEQ ID NO: 4 - DNA, 70 bp, Artificial sequence
  vector
ggccctgcagctgaaggctttgaggctnnnnnngcannngccnnnnnngctgtgccatctaagtggcccc

SEQ ID NO: 5 - protein, 20 aa, Artificial sequence
  synthetic peptide motif
PAAEGFEAXXAXAXXAVPSK

SEQ ID NO: 6 - DNA, 79 bp, Artificial sequence
  vector
cagccctgcagctgaaggctttgaggctnnnnnngcannngccnnnnnngctgtgccatctaaggtagcgacactagtg

SEQ ID NO: 7 - protein, 26 aa, Artificial sequence
  synthetic peptide motif
SPAAEGFEAXXAXAXXAVPSKVATLV

SEQ ID NO: 8 - protein, 44 aa, Artificial sequence
  synthetic peptide motif
KHWEPEIPAPGGGTAQLKKKLQALKKKNAQLKWKLQALKKKLAQ

SEQ ID NO: 9 - protein, 78 aa, Artificial sequence
  synthetic peptide motif
VWKAQSESAGGRTAQLEKELQALEKENAQLEWELQALEKELAQASGGGFMFGHVVNFVIILIVILFLYCMIRNRNRQY

SEQ ID NO: 10 - protein, 13 aa, Artificial sequence
  synthetic peptide motif
FEAQRARAARAVD

SEQ ID NO: 11 - protein, 13 aa, Artificial sequence
  synthetic peptide motif
FEAQKAKANKAVD

SEQ ID NO: 12 - protein, 13 aa, Artificial sequence
  synthetic peptide motif
FEARCAKASTAVD

SEQ ID NO: 13 - protein, 13 aa, Artificial sequence
  synthetic peptide motif
FEAFPAKALRAVD

SEQ ID NO: 14 - protein, 13 aa, Artificial sequence
  synthetic peptide motif
FEASKASAAVAVD

SEQ ID NO: 15 - protein, 13 aa, Artificial sequence
  synthetic peptide motif
FEARLASAGKAVD

SEQ ID NO: 16 - protein, 12 aa, Artificial sequence
  synthetic peptide motif
FEAQRARAARAV

SEQ ID NO: 17 - protein, 12 aa, Artificial sequence
  synthetic peptide motif
FEAQKAKANKAV

SEQ ID NO: 18 - protein, 12 aa, Artificial sequence
  synthetic peptide motif
FEAQGALANIAV

SEQ ID NO: 19 - protein, 12 aa, Artificial sequence
  synthetic peptide motif
FEALYAKALTAV

SEQ ID NO: 20 - protein, 12 aa, Artificial sequence
  synthetic peptide motif
FEARCAKASTAV

SEQ ID NO: 21 - protein, 12 aa, Artificial sequence
  synthetic peptide motif
FEAFMARAKAAV

SEQ ID NO: 22 - protein, 12 aa, Artificial sequence
  synthetic peptide motif
FEAQTAKARGAV

SEQ ID NO: 23 - protein, 12 aa, Artificial sequence
  synthetic peptide motif
FEALPARAAAAV

SEQ ID NO: 24 - protein, 12 aa, Artificial sequence
  synthetic peptide motif
FEAHTALAPRAV

SEQ ID NO: 25 - protein, 12 aa, Artificial sequence
  synthetic peptide motif
FEASLARARSAV

SEQ ID NO: 26 - protein, 12 aa, Artificial sequence
  synthetic peptide motif
FEAYTARARTAV

SEQ ID NO: 27 - protein, 12 aa, Artificial sequence
  synthetic peptide motif
FEATTARALTAV

SEQ ID NO: 28 - protein, 12 aa, Artificial sequence
  synthetic peptide motif
FEAEKAKALTAV

SEQ ID NO: 29 - protein, 12 aa, Artificial sequence
  synthetic peptide motif
FEAQVAHALPAV

SEQ ID NO: 30 - protein, 12 aa, Artificial sequence
  synthetic peptide motif
FEAFPAKALRAV

SEQ ID NO: 31 - protein, 12 aa, Artificial sequence
  synthetic peptide motif
FEALSAKANTAV

SEQ ID NO: 32 - protein, 12 aa, Artificial sequence
  synthetic peptide motif
FEAREAKALAAV

SEQ ID NO: 33 - protein, 12 aa, Artificial sequence
  synthetic peptide motif
FEAALARAVPAV

SEQ ID NO: 34 - protein, 12 aa, Artificial sequence
  synthetic peptide motif
FEASKASAAVAV

SEQ ID NO: 35 - protein, 12 aa, Artificial sequence
  synthetic peptide motif
FEARLASAGKAV

SEQ ID NO: 36 - protein, 12 aa, Artificial sequence
  synthetic peptide motif
FEAERARAASAV

SEQ ID NO: 37 - protein, 12 aa, Artificial sequence
  synthetic peptide motif
FEARTAHARNAV

SEQ ID NO: 38 - protein, 12 aa, Artificial sequence
  synthetic peptide motif
FEAPYAQAPHAV

SEQ ID NO: 39 - protein, 13 aa, Artificial sequence
  synthetic peptide motif
FEAQTAKARGAVD

SEQ ID NO: 40 - protein, 13 aa, Artificial sequence
  synthetic peptide motif
FEALPARAAAAVD

SEQ ID NO: 41 - DNA, 161 bp, Artificial sequence
  vector
accctgagatggggcaagcagtccactaggggtggagctagcggcggaggtttcatgtttggtcatgtagttaactttgtaattatattaattgtgattttatttttatactgtatgattagaaaccgtaatagacaatattaaccaacatgcggggatcc

SEQ ID NO: 42 - protein, 47 aa, Artificial sequence
  synthetic peptide motif
TLRWGKQSTRGGASGGGFMFGHVVNFVIILIVILFLYCMIRNRNRQY

SEQ ID NO: 43 - DNA, 156 bp, Artificial sequence
  vector
cgacagaggacagaggccatggctcgctcggtgaccctagtctttctggtgcttgtctcactgaccggcttgtatgctcgcggaccgggcagagccttcgtgaccatcggaggtggcgggtccggaggtggttctggtggaggttcgatccagaaa

SEQ ID NO: 44 - protein, 46 aa, Artificial sequence
  synthetic peptide motif
MARSVTLVFLVLVSLTGLYARGPGRAFVTIGGGGSGGGSGGGSIQK

SEQ ID NO: 45 - DNA, 65 bp, Artificial sequence
  vector
gggggatcgatcctctagagtcgagcaagaaaataaaacgccaaacgcgttggagtcttgtgtgc

SEQ ID NO: 46 - DNA, 90 bp, Artificial sequence
  vector
gtgcttgtctcactgaccggcttgtatgctnnnggaccgnnncggnnnnnnnnnctcggaggtggcgggtccggaggtggttctggtgga

SEQ ID NO: 47 - protein, 30 aa, Artificial sequence
  synthetic peptide motif
VLVSLTGLYAXGPXRXXXLGGGGSGGGSGG

SEQ ID NO: 48 - DNA, 93 bp, Artificial sequence
  vector
gtgcttgtctcactgaccggcttgtatgctnnnggaccgnnncggnnnnnnnnnnnnctcggaggtggcgggtccggaggtggttctggtgga

SEQ ID NO: 49 - protein, 31 aa, Artificial sequence
  synthetic peptide motif
VLVSLTGLYAXGPXRXXXXLGGGGSGGGSGG

SEQ ID NO: 50 - protein, 9 aa, Artificial sequence
  synthetic peptide motif
TGPTRWCRL

SEQ ID NO: 51 - protein, 9 aa, Mus musculus
AGATRWCRL

SEQ ID NO: 52 - DNA, 39 bp, Artificial sequence
  vector
attaccctgttatccctaggatcttggagataattaaaa

SEQ ID NO: 53 - DNA, 51 bp, Artificial sequence
  vector
tacaagtaaagcggccgctctaactataacggtcctaaggtagcgacacta

SEQ ID NO: 54 - protein, 6 aa, Artificial sequence
  synthetic peptide motif
XKVATL

SEQ ID NO: 55 - DNA, 41 bp, Artificial sequence
  vector
gattacccagttatctggagataattaaaatgataaccatc

SEQ ID NO: 56 - DNA, 68 bp, Artificial sequence
  vector
cagccccgggactgaaggcnnntttnnnnnnccgnnngccnnnnnngctgtgccatctaagtgggcga

SEQ ID NO: 57 - protein, 21 aa, Artificial sequence
  synthetic peptide motif
SPGTEGXFXXPXAXXAVPSKX

SEQ ID NO: 58 - DNA, 40 bp, Artificial sequence
  vector
attaccctgttatctggagataattaaaatgataaccatc

SEQ ID NO: 59 - DNA, 73 bp, Artificial sequence
  vector
cagccccgggactgaaggcnnntttnnnnnnccgnnngccnnnnnngctgtgccatctaaggtagcgacacta

SEQ ID NO: 60 - protein, 24 aa, Artificial sequence
  synthetic peptide motif
SPGTEGXFXXPXAXXAVPSKVATL

(19) United States
(12) Patent Application Publication
(10) Pub. No.: US 2004/0110253 A1
(43) Pub. Date: Jun. 10, 2004

(54) Method for identifying MHC-presented peptide epitopes for T cells

(76) Inventors: John W. Kappler, Denver, CO (US); Frances G. Crawford, Louisville, CO (US); Philippa Marrack, Denver, CO (US)

Correspondence Address: SHERIDAN ROSS PC, 1560 BROADWAY, SUITE 1200, DENVER, CO, 80202

(21) Appl. No.: 10/640,881

(22) Filed: Aug. 13, 2003

Related U.S. Application Data
(60) Provisional application No. 60/403,291, filed on Aug. 13, 2002.

(51) Int. Cl. C07H 21/04; C12N 5/06; C12N 15/86; C07K 14/74

(52) U.S. Cl. 435/69.1; 435/456; 435/348; 530/350; 536/23.5

(57) ABSTRACT

Described are three basic components: (1) methods for the display of functional MHC molecules with covalently attached antigenic peptides on the surface of baculovirus and baculovirus infected insect cells; (2) methods for the identification and physical isolation of baculovirus or baculovirus infected insect cells bearing a displayed MHC/peptide combination that is recognized by a particular T cell antigen receptor; and (3) methods for producing libraries of baculovirus or baculovirus infected insect cells displaying a particular MHC molecule and many different potential antigenic peptides.

### CROSS-REFERENCE TO RELATED APPLICATIONS

This application claims priority under 35 U.S.C. § 119(e) from U.S. Provisional Application Serial No. 60/403,291, filed Aug. 13, 2002, entitled “Method for Identifying MHC-Presented Peptide Epitopes for T Cells”. The entire disclosure of U.S. Provisional Application No. 60/403,291 is incorporated herein by reference.

### FIELD OF THE INVENTION

This invention generally relates to a recombinant baculovirus expression vector for expression of functional MHC-peptide molecules, to a method to produce libraries of functional MHC-peptide molecules displayed on the surface of baculovirus and baculovirus-infected cells, and to a method for identifying baculovirus or baculovirus-infected cells that display an MHC-peptide complex that is recognized by a specific T cell receptor.

### BACKGROUND OF THE INVENTION

The identification of peptide epitopes associated with particular αβ T cell receptors is often still a bottle neck in studying T cells and their antigenic targets in, for example, autoimmunity, hypersensitivity, and cancer. In many clinical situations, when pathological T cells are identified, only the major histocompatibility complex (MHC), but not the specific peptide portion of the antigen that is recognized by the T cell, is known. Having a rapid method to identify these peptides would aid in the identification of the protein source of the antigens driving the T cell responses. These peptides would help also in creating tools to monitor the frequency and functional state of the T cells as well as the development of therapeutic reagents to control them.

A direct genetic or biochemical attack on this problem can be successful, especially with MHC Class I presented peptides. For example, direct screening of cDNA libraries has resulted in the identification of a number of tumor antigens (Van Der Bruggen et al., 2002, Immunol. Rev. 188:51-64). Identification of the antigenic peptide in a mix of peptides stripped from MHC molecules isolated from antigen presenting cells (APCs) has sometimes been possible using a combination of a biological assay, peptide fractionation and peptides sequencing (Guimezanes et al., 2001, Eur. J. Immunol. 31:421-432). However, this method is extremely labor intensive and depends on relatively high peptide frequency in the mix and a very sensitive bioassay. These conditions are not always achievable, especially with peptides presented by MHC Class II in which peptide loading of surface MHC may require peptide concentrations orders of magnitude higher than those required for MHC Class I loading.

The reward for the labor involved in identifying peptide epitopes directly can often be the identification of the protein source of the peptide especially as the sequencing of the genomes of many organisms approaches completion. However, in many situations, rather than identifying this precise peptide epitope, it is sufficient to identify a peptide “mimotope.” Mimotopes can be defined as peptides that are different in sequence from the actual peptide recognized in vivo, but which are nevertheless capable of binding to the appropriate MHC molecules to form a ligand that can be recognized by the αβTCR in question. These peptides can be very useful for studying the T cell in vitro, altering the immunological state of the T cell in vivo (Hogquist et al., 1994, Cell, 76:17-27), vaccine development (Partidos, 2000, Curr Opin Mol Ther 2:74-79) and potentially in preparing multimeric fluorescent peptide/MHC complexes for tracking T cells in vivo.

Mimotopes can be identified in randomized peptide libraries that can be screened for presentation by a particular MHC molecule to the relevant T cell (Gavin et al., 1994, Eur J. Immunol, 24:2124-2133; Linnemann et al., 2001, Eur J Immunol, 31:156-165; Sung et al., 2002, J Comput Biol, 9:527-539), reviewed in (Hiemstra et al., 2000, Curr Opin Immunol, 12:80-84) and (Liu et al., 2003, Exp Hematol, 31:11-30). Thus far, strategies for screening these types of libraries have involved individual testing of pools of peptides from the library and then either deduction of the mimotope sequence from the pattern of responses or sequential reduction in the size of the pool until a single peptide emerges (since the peptides are not linked to the DNA that encodes them, they cannot be amplified). There are several limitations to this type of approach. Again, a very sensitive T cell bioassay is needed in which the activity of the correct stimulating peptide is not masked by competition with the other peptides in the pool. Also, an APC that expresses the relevant MHC molecule, but not the relevant peptide, must be found or constructed. Finally, because the screen relies on T cell stimulation, only agonist mimotope peptides are identified. This method is very time consuming and costly. Because of the labor involved in this type of screens, these libraries are usually much smaller than those possible with phage display.

In other applications, another powerful library method has been sequential enrichment/expansion of a displayed library of protein/peptide variants by direct ligand/receptor binding, e.g. using bacterial phage or yeast (also reviewed in Liu et al., 2003, Exp Hematol, 31:11-30). These methods have not yet been developed for the routine identification of T cell antigen mimotopes, because of the lack of a suitable system for the display of MHC/peptides or for screening via αβTCR binding using these organisms.

Therefore, there is a need in the art for a rapid, effective, and inexpensive method for screening large numbers ofpeptides and selecting those that are MHC-presented epitopes for T cells.

### SUMMARY OF THE INVENTION

One embodiment of the present invention relates to a recombinant baculovirus expression vector for expression offunctional MHC-peptide molecules. The vector includes a baculovirus genome comprising: (a) a first nucleic acid sequence inserted into a first baculovirus structural gene at a position under control of a promoter for the first baculovirus structural gene, wherein the first nucleic acid sequence encodes at least a portion of the extracellular domains of the a chain of a major histocompatibility complex (MHC) Class I molecule or at least a portion of the extracellular domains of the α chain of a MHC Class II molecule; (b) a second nucleic acid sequence inserted into a second baculovirus structural gene at a position under control of a promoter for the second baculovirus structural gene, wherein the second nucleic acid sequence encodes at least a portion of the extracellular domains of: (i) a β2-microglobulin (β2m) chain of a MHC Class I molecule if the first nucleic acid sequence encodes at least a portion of the extracellular domains of the α chain of a MHC Class I molecule; or (ii) a β chain of a MHC Class II molecule if the first nucleic acid sequence encodes at least a portion of the extracellular domains of the α chain of a MHC Class II molecule; (c) a third nucleic acid sequence encoding an MHC-binding peptide; (d) a fourth nucleic acid sequence encoding a peptide linker, wherein the third nucleic acid sequence encoding the MHC-binding peptide is connected to the 5′ end of the first or second nucleic acid sequence by the fourth nucleic acid sequence; and (e) a fifth nucleic acid sequence encoding at least a transmembrane region of a membrane protein, wherein the first or the second nucleic acid sequence is inserted into the baculovirus genome in frame with the fifth nucleic acid sequence, the fifth nucleic acid sequence being located after the 3′ end of the first or second nucleic acid sequence. The portion of the extracellular domains of the α chain of the MHC Class I molecule and the portion of the extracellular domains of the β2m chain of the MHC Class I molecule, or the portion of the extracellular domains of the α chain of the MHC Class II molecule and the portion of the extracellular domains of the β chain of the MHC Class II molecule, form a peptide binding groove of an MHC molecule, and wherein the MHC-binding peptide comprises a sequence of amino acids that binds to the peptide binding groove.

In one aspect, the first nucleic acid sequence encodes at least a portion of the extracellular domains of the α chain of a MHC Class I molecule, and wherein the second nucleic acid sequence encodes at least a portion of the extracellular domains of a β2m chain of a MHC Class I molecule. In this aspect, the third nucleic acid sequence encoding the MHC-binding peptide can be connected to the 5′ end of the second nucleic acid sequence encoding at least a portion of the extracellular domains of a β2m chain of a MHC Class I molecule by the fourth nucleic acid sequence encoding a peptide linker.

In another aspect, the first nucleic acid sequence encodes at least a portion of the extracellular domains of the α chain of a MHC Class II molecule, and wherein the second nucleic acid sequence encodes at least a portion of the extracellular domains of a β chain of a MHC Class II molecule. In this aspect, the third nucleic acid sequence encoding the MHC-binding peptide can be connected to the 5′ end of the second nucleic acid sequence encoding at least a portion of the extracellular domains of a β chain of a MHC Class II molecule by the fourth nucleic acid sequence encoding a peptide linker.

In one aspect, the fifth nucleic acid sequence can include, but is not limited to, a nucleic acid sequence encoding at least the transmembrane portion of a membrane protein chosen from: baculovirus envelope protein gp64, MHC Class I, MHC Class II, and p26. In one aspect, the fifth nucleic acid sequence encodes at least the transmembrane portion of baculovirus envelope protein gp64. In another aspect, the fifth nucleic acid sequence encodes a full-length gp64. In another aspect, the fifth nucleic acid sequence encodes only the transmembrane portion and cytoplasmic tail of gp64.

In one aspect, the first nucleic acid sequence further comprises, 3′ of the nucleic acid sequence encoding the extracellular domains of the α chain of an MHC molecule, a nucleic acid sequence encoding a basic leucine zipper dimerization helix.

In another aspect, the second nucleic acid sequence further comprises, 3′ of the nucleic acid sequence encoding the extracellular domains of the β chain of a Class II MHC molecule or the Class I β2m molecule, a nucleic acid sequence encoding an acidic leucine zipper dimerization helix.

In one aspect, the peptide linker encoded by the fourth nucleic acid molecule comprises at least about 8 amino acid residues, wherein the linker facilitates the binding of the MHC-binding peptide to the peptide binding groove of the MHC molecule. In one aspect, the MHC-binding peptide is from a library of candidate antigenic peptides, wherein the each of the peptides in the library comprises conserved amino acids in a specific sequence sufficient to enable the peptide to bind to the peptide binding groove of the MHC molecule that is encoded by the vector. In another aspect, the MHC-binding peptide is from a library of candidate antigenic peptides, wherein each of the peptides in the library comprises between about 4 and 5 conserved amino acids in a specific sequence sufficient to enable the peptide to bind to the peptide binding groove of the MHC molecule that is encoded by the vector. In another aspect, the MHC-binding peptide is from a library of candidate antigenic peptides representing from between about 10³ and about 10⁹ different candidate antigenic peptides.

Another embodiment of the invention relates to a recombinant baculovirus comprising the recombinant baculovirus expression vector as described above, wherein the recombinant baculovirus expresses and displays on its surface a functional MHC-peptide molecule encoded by the vector. Another embodiment of the invention relates to a population of cells infected with such a recombinant baculovirus, wherein the cells display the functional MHC-peptide molecules expressed by the baculovirus on their surfaces.

Yet another embodiment of the present invention relates to a recombinant insect cell that displays on its surface a functional MHC-peptide molecule. The recombinant insect cell has been transfected with recombinant nucleic acid molecules that encode at least the extracellular domains of an MHC molecule, the recombinant nucleic acid molecules comprising: (i) a first nucleic acid sequence operatively linked to an expression control sequence, wherein the first nucleic acid sequence encodes at least a portion of the extracellular domains of the α chain of a major histocompatibility complex (MHC) Class I molecule or at least a portion of the extracellular domains of the α chain of a MHC Class II molecule; and (ii) a second nucleic acid sequence operatively linked to an expression control sequence under control of a baculovirus promoter and enhancer, wherein the second nucleic acid sequence encodes at least a portion of the extracellular domains of: (1) a β2-microglobulin (β2m) chain of a MHC Class I molecule if the first nucleic acid sequence encodes at least a portion of the extracellular domains of the α chain of a MHC Class I molecule; or (2) a β chain of a MHC Class II molecule if the first nucleic acid sequence encodes at least a portion of the extracellular domains of the α chain of a MHC Class II molecule. The portion of the extracellular domains of the α chain of the MHC Class I molecule and the portion of the extracellular domains of the β2m chain of the MHC Class I molecule, or the portion of the extracellular domains of the α chain of the MHC Class II molecule and the portion of the extracellular domains of the β chain of the MHC Class II molecule, form a peptide binding groove of an MHC molecule. The recombinant insect cell has also been infected with a recombinant baculovirus comprising a third nucleic acid sequence under control of a baculovirus promoter and comprising a signal sequence, wherein the third nucleic acid sequence encodes an MHC-binding peptide, wherein the MHC-binding peptide comprises a sequence of amino acids that binds to the peptide binding groove of the MHC Class I molecule or the MHC Class II molecule.

Yet another embodiment of the invention relates to a method for production of libraries of functional MHC-peptide molecules displayed on the surface of baculovirus and baculovirus-infected cells. The method includes a first step of: (a) producing a population of recombinant baculoviruses by introducing into the genome of the baculoviruses: (i) a first nucleic acid sequence encoding at least a portion of the extracellular domains of the α chain of a major histocompatibility complex (MHC) Class I molecule or at least a portion of the extracellular domains of the α chain of a MHC Class II molecule, wherein the first nucleic acid sequence is introduced into the baculovirus genome at a position under control of a promoter for a first baculovirus structural gene; (ii) a second nucleic acid sequence encoding at least a portion of the extracellular domains of: (1) a β2-microglobulin (β2m) chain of a MHC Class I molecule if the first nucleic acid sequence encodes at least a portion of the extracellular domains of the α chain of a MHC Class I molecule; or (2) a β chain of a MHC Class II molecule if the first nucleic acid sequence encodes at least a portion of the extracellular domains of the α chain of a MHC Class II molecule; (iii) a third nucleic acid sequence encoding a candidate antigenic peptide, wherein the candidate antigenic peptide is randomly produced from a possible library of candidate antigenic peptides so that each baculovirus in the population may express a different candidate antigenic peptide, wherein each of the peptides in the library comprises: (1) conserved amino acid residues at specific positions in the sequence sufficient to enable the peptide to bind to the MHC molecule; and (2) randomly generated amino acid residues in the remaining positions in the sequence; (iv) a fourth nucleic acid sequence encoding a peptide linker, wherein the third nucleic acid sequence encoding a candidate antigenic peptide is connected to the first or second nucleic acid sequence by the fourth nucleic acid sequence; (v) a fifth nucleic acid sequence encoding at least the transmembrane portion of a membrane protein, the membrane protein-encoding sequence being in frame with and located after the 3′ end of the first or second nucleic acid sequence. The second nucleic acid sequence is introduced into the baculovirus genome at a position under control of a promoter for a second baculovirus structural gene, and the portion of the extracellular domains of the α chain of the MHC Class II molecule and the portion of the extracellular domains of the α chain of the Class II MHC molecule, or the portion of the extracellular domains of the α chain of the Class I MHC molecule and the portion of the extracellular domains of the β2m chain of the Class I MHC molecule, respectively, form a peptide binding groove. The third nucleic acid sequence is introduced into the baculovirus genome before the 5′ end of the first or second nucleic acid sequence. The method includes an additional step of: (b) expressing the nucleic acid sequences of (i)-(v) on the surface of each of the baculoviruses in the population, wherein expression of the nucleic acid sequences of (i)-(v) results in the production of at least a portion of an MHC molecule which is covalently linked to the candidate antigenic peptide expressed by the given baculovirus via the peptide linker, and wherein the candidate antigenic peptide is bound to the peptide binding groove of the MHC molecule, thereby forming a library of MHC-peptide molecules displayed on the surface of baculoviruses, the library representing multiple different candidate antigenic peptides.

In one aspect, the method includes an additional step of infecting cells with the recombinant baculoviruses, so that an MHC-peptide molecule from the library of MHC-peptide molecules is displayed on the surface of each of the cells infected by the baculovirus. In one aspect, the fifth nucleic acid sequence encodes at least the transmembrane portion of baculovirus envelope protein gp64. In another aspect, each of the peptides in the library comprises between about 4 and 5 conserved amino acids in a specific sequence sufficient to enable the peptide to bind to the MHC molecule. In another aspect, the nucleic acid sequences are introduced into the baculovirus genome using an E. coli transfer plasmid. In another aspect, the nucleic acid sequences are introduced into the baculovirus genome by direct cloning of the sequences into the genome. In one aspect, the library of candidate antigenic peptides represents from about 10³ to about 10⁹ different candidate antigenic peptides.

Another embodiment of the invention relates to a library of functional MHC-peptide molecules displayed on the surface of baculovirus or baculovirus-infected cells produced by the method described above.

Yet another embodiment of the invention relates to a population of cells infected with the recombinant baculoviruses produced by the method described above, wherein an MHC-peptide molecule from the library of MHC-peptide molecules is displayed on the surface of each of the cells infected by the baculovirus.

Another embodiment of the invention relates to a method for identifying baculovirus or baculovirus-infected cells that display an MHC-peptide complex that is recognized by a specific T cell receptor. The method includes the steps of: (a) providing baculoviruses or baculovirus-infected cells that display on the baculoviral surface or cell surface, respectively, at least one MHC-peptide complex, wherein the complex comprises: (i) at least a portion of an MHC molecule sufficient to form a peptide binding groove; and (ii) a candidate antigenic peptide that is covalently linked to the MHC molecule by a peptide linker and which is bound to the peptide binding groove of the MHC molecule, wherein the candidate antigenic peptide is from a library of candidate antigenic peptides, wherein each of the peptides in the library comprises conserved amino acids in a specific sequence sufficient to enable the peptide to bind to the MHC molecule; (b) contacting the baculoviruses or baculovirus-infected cells with a target T cell receptor; and (c) selecting baculoviruses or baculovirus-infected cells that bind to the target T cell receptor.

In one aspect of this embodiment, the method includes the additional steps of: (d) isolating the selected baculoviruses or baculoviruses from the selected baculovirus-infected cells of step (c); (e) infecting previously uninfected host cells with the isolated baculoviruses of (d) to produce baculoviruses or baculovirus-infected cells enriched for MHC-peptide complexes that bind to the target T cell receptor; (f) contacting the baculoviruses or baculovirus-infected cells from (e) with the target T cell receptor; and (g) selecting baculoviruses or baculovirus-infected cells that bind to the target T cell receptor. In another aspect, the method further includes the step of isolating the selected baculoviruses or the baculoviruses from the selected baculovirus-infected cells of step (g) and repeating steps (e)-(g) at least one additional time to isolate and identify an MHC-peptide complex that binds to the target T cell receptor.

In one aspect of this embodiment, the target T cell receptor is labeled with a detectable label. In one aspect, the target T cell receptor is expressed on the surface of a cell. In one aspect, the target T cell receptor is soluble and immobilized on a substrate. In another aspect, the library of candidate antigenic peptides represents from about 10³ to about 10⁹ different candidate antigenic peptides. In another aspect, the target T cell receptor is from a patient with a T cell-mediated disease.

### BRIEF DESCRIPTION OF THE DRAWINGS OF THE INVENTION

FIG. 1A is a schematic drawing showing one method to display functional MHC Class II using baculovirus, including incorporation of full length baculoviral envelop protein, gp64.

FIG. 1B is a schematic drawing showing one method to display functional MHC Class II using baculovirus, including incorporation of only the transmembrane and cytoplasmic tail of gp64.

FIG. 1C is a schematic drawing showing one method to display functional MHC Class II using baculovirus, including incorporation of basic and acidic leucine zipper dimerization helices.

FIG. 2 is a schematic drawing showing the display of MHC-peptide complexes on the baculovirus surface or infected insect cell surface.

FIG. 3 is a graph showing the detection of displayed IA^{b}-p3K on the surface of infected SF9 insect cells.

FIG. 4 is a graph showing the recognition by T cells of known specificity of functional IA^{b}-p3K displayed on infected SF9 insect cells.

FIG. 5 is a schematic drawing showing methods of identifying a displayed MHC-peptide complex recognized by a specific T cell receptor using the method of the invention.

FIG. 6 is a graph showing the use of immobilized soluble T cell receptor to capture baculovirus displaying IA^{b}-p3K-gp64 complexes that are recognized by the T cell receptor.

FIG. 7 is a schematic drawing showing the use of fluorescently labeled soluble T cell receptor to capture insect cells displaying MHC-peptide complexes that are bound by the receptor.

FIG. 8A is a schematic drawing showing the configuration of baculovirus DNA for construction of an IA^{b}-peptide library by direct cloning in baculovirus DNA (the nucleotide sequence showing the site for SbfI is represented by SEQ ID NO:1; the nucleotide sequence showing the site for CeuI is represented by SEQ ID NO:2; the amino acid sequence of the beginning of the linker peptide is represented by SEQ ID NO:3).

FIG. 8B is a schematic drawing showing the configuration of the randomized fragment for construction of an IA^{b}-peptide library by direct cloning in baculovirus DNA (nucleotide sequence depicted is represented by SEQ ID NO:4; peptide sequence depicted is represented by SEQ ID NO:5).

FIG. 8C is a schematic drawing showing the configuration of the randomized fragment inserted into the baculovirus DNA for construction of an IA^{b}-peptide library by direct cloning in baculovirus DNA (nucleotide sequence depicted is represented by SEQ ID NO:6; peptide sequence depicted is represented by SEQ ID NO:7).

FIG. 9A is a schematic drawing showing the construct for the modified α chain of IA^{b} used in Example 1 (sequence depicted is represented by SEQ ID NO:8).

FIG. 9B is a schematic drawing showing the construct for the modified β chain of IA^{b} used in Example 1 (sequence depicted is represented by SEQ ID NO:9).

FIG. 10A is graph showing results of peptide screening of B3K-06 TcR with representative baculovirus clones expressing the IA^{b}-peptide complex (each of B23, B 17, B13, and B9 is represented by positions 1-12 of SEQ ID NO:10; p3K is represented by positions 1-12 of SEQ ID NO:11).

FIG. 10B is graph showing results of peptide screening of YAe-62 TcR with representative baculovirus clones expressing the IA^{b}-peptide complex (Y2=positions 1-12 of SEQ ID NO:12; Y28=positions 1-12 of SEQ ID NO:13; Y52=positions 1-12 of SEQ ID NO:14; Y14=positions 1-12 of SEQ ID NO:15; p3K=positions 1-12 of SEQ ID NO:11).

FIG. 11A is a schematic drawing showing the construct for the modified Class I heavy chain of D^{d} used in Example 2 (nucleotide sequence depicted is represented by SEQ ID NO:41; amino acid sequence depicted is represented by SEQ ID NO:42).

FIG. 11B is a schematic drawing showing the construct for the modified Class I β2 microglobulin chain used in Example 2 (nucleotide sequence depicted is represented by SEQ ID NO:43; amino acid sequence depicted is represented by SEQ ID NO:44).

FIG. 12A is a graph showing expression of D^{d} on the surface of SF9 cells infected with D^{d}-pHIV expressing baculovirus.

FIG. 12B is a graph showing production of IL-2 by B4.2.3 in response to SF9 cells infected with D^{d}-pHIV expressing baculovirus.

FIG. 13A is a schematic drawing showing the construction of a modified β2m gene of D^{d}-pHIV disrupted by a sequence encoding enhanced GFP (eGFP) with a TAA termination codon to prevent read through into the β2m gene.

FIG. 13B is a schematic drawing showing the forward oligonucleotide primer used to construct a PCR fragment that encoded peptides that could bind to D^{d} (nucleotide sequence depicted is represented by SEQ ID NO:45).

FIG. 13C is schematic drawing showing the reverse oligonucleotide primers used to construct a PCR fragment that encoded peptides that could bind to D^{d} (nucleotide sequence depicted for 9mer is represented by SEQ ID NO:46; amino acid sequence depicted for 9mer is represented by SEQ ID NO:47; nucleotide sequence depicted for 10mer is represented by SEQ ID NO:48; amino acid sequence depicted for 10mer is represented by SEQ ID NO:49).

FIG. 13D is a schematic drawing showing the structure of the β2m construct after replacement of the GFP gene with the PCR fragments.

FIG. 14A is a graph showing IL-2 produced by T cell 3DT-52.5 in response to ICAM+/B7+ SF9 cells infected with baculovirus expressing D^{d} tethered to either pHIV or the αβTCR identified peptide, TGPTRWCRL (SEQ ID NO:50).

FIG. 14B is a graph showing IL-2 produced by T cell 3CDT-52.5 in response to (1) P815 plus a bound unknown self-peptide and 2) LKD8, alone, or in the presence of the library derived peptide, TGPTRWCRL (SEQ ID NO:50), or a peptide derived from the spin protein, AGATRWCRL (SEQ ID NO:51).

FIG. 15A is a schematic drawing showing the baculovirus construct encoding the genes for a displayed version of the MHC Class II IA^{b} molecule which is a recipient DNA for the IA^{b} libraries (nucleotide sequence showing the site for Scel and a portion of the pH promoter is represented by SEQ ID NO:52; the nucleotide sequence showing the site for CeuI and the linker is represented by SEQ ID NO:53; the amino acid sequence depicted for the linker portion is represented by SEQ ID NO:54).

FIG. 15B is a schematic drawing showing a PCR fragment encoding the polyhedrin promoter, the IA^{b} beta chain signal peptide and an IA^{b} binding peptide in which codons for six amino acids predicted to be surface exposed in the IA^{b}-peptide complex were randomized (nucleotide sequence showing the BstXI site and a portion of the pH promoter is represented by SEQ ID NO:55; nucleotide sequence showing the BstXI site and sequence encoding a portion of the signal peptide, randomized peptide and linker is represented by SEQ ID NO:56; amino acid sequence depicted for the portion of the signal peptide, randomized peptide and linker is represented by SEQ ID NO:57).

FIG. 15C is a schematic drawing showing the final baculovirus construct DNA for the IA^{b} library (nucleotide sequence showing a portion of the pH promoter is represented by SEq ID NO:58; nucleotide sequence showing the sequence encoding a portion of the signal peptide, randomized peptide, and linker is represented by SEQ ID NO:59; amino acid sequence depicting a portion of the signal peptide, randomized peptide, and linker is represented by SEQ ID NO:60).

FIG. 15D is a schematic drawing showing the baculovirus recipient DNA for MHC Class I libraries.

### DETAILED DESCRIPTION OF THE INVENTION

The present invention generally relates to a method to identify peptides that can combine with a known major histocompatibility complex (MHC) molecule to create a ligand that is recognized by a known T cell receptor. More specifically, the present invention uses baculovirus to produce a very large library of MHC molecules with covalently or non-covalently attached randomized variant peptides. The construction allows the surface display of the MHC/peptide complex on the surface of both the baculovirus and the baculovirus infected insect cells. For a given T cell, either virus or virus-infected cells encoding the correct MHC/peptide complexes can be selected and purified based on their direct binding to the T cell receptor expressed by the T cell or to a soluble recombinant αβ T cell receptor prepared from the T cell. The sequence of the peptides can be deduced from the DNA sequences of the purified viruses. As discussed above, the peptides are then useful as tools to aid in the identification of the protein source of the antigens driving the T cell responses, as well as for creating tools to monitor the frequency and functional state of the T cells and for developing therapeutic reagents to regulate the T cells.

The present invention has all of the advantages of phage display without the disadvantages. Because the library of random peptides are produced genetically with PCR generated DNA fragments, very large libraries can be achieved. A large variety of MHC molecules from a both mouse and man have been produced with bound covalent peptides using baculovirus. Whether displayed on the baculovirus or the infected insect cell surface, these MHC/peptide complexes are recognized and bound by T cells and soluble αβ T cell receptors. Therefore the complete library can be “fished” by direct binding to a T cell or soluble T cell receptor (i.e., the “bait”).

This method was developed using IA^{b} as the displayed MHC Class II molecule carrying the peptide library (see Example 1). However, using the same strategy, the inventors have successfully displayed numerous other MHC Class II molecules, such as murine IE^{k} and human DR52c (data not shown). Moreover, the inventors (White et al., 1999, J Immunol, 162:2671-2676) and others (Mottez et al., 1995, J Exp Med, 181:493-502; Uger and Barber, 1998, J Immunol, 160:1598-1605) have shown that peptides can be tethered to MHC Class I molecules via the N-terminus of either β2M or the heavy chain, making the new approach disclosed herein feasible for searching for MHC Class I bound peptide mimotopes as well. In preliminary experiments, it has been successfully used to display on the surface of SF9 cells the mouse MHC Class I molecule, D^{d}, with a β2m tethered peptide from HIV gp120 (data not shown). Given that baculovirus has been such a successful expression system for many different types of complex eukaryotic proteins that express or assembly poorly in E. coli, the novel method of the present invention may have broad applications to other receptor/ligand systems.

As opposed to methods that use T cell activation as the peptide screening method, an advantage of display methods that use flow cytometry for screening and enrichment is that the strength of binding of receptor and ligand can be estimated and manipulated. In the results reported herein, by limiting the analysis to insect cells bearing a particular level of MHC/peptide, a uniform level of αβTCR binding was seen for an individual peptide sequence, but the strength of binding varied over two orders of magnitude for different peptides, presumably reflecting the relative affinity of the receptor for different IA^{b}/peptide combinations. Thus, depending on whether one was interested in high or low affinity ligands for the αβTCR, one could enrich for peptides with these properties directly during the screening of the library. Such an approach has been used with antibody (Boder and Wittrup, 2000, Methods Enzymol, 328:430-444) and αβTCR (Shusta et al., 2000, Nat Biotechnol, 18:754-759) variants displayed on yeast to select directly for receptors with increased affinity.

One of the surprising results in the present inventors' studies was the relationship between the strength of αβTCR binding to a particular MHC/peptide combination and the subsequent level of IL-2 secretion seen from the T cell responding to this combination. While IL-2 secretion was seen only for that set of peptides that yielded IA^{b}-peptide complexes with the highest apparent affinities, there was a great deal of variation in the amount of IL-2 produced by complexes with very similar apparent affinities. One possibility is that the baculovirus produced soluble αβTCR used in these studies differs subtly in specificity from the αβTCR on the surface of the T cell hybridoma, e.g. due to differences in glycosylation or because of the effects of CD3 or CD4. However, and without being bound by theory, a more interesting possibility is that this variation in stimulation is related to the phenomenon of altered peptide ligands in which amino acid variants of fully immunogenic peptides only partially activate or even angergize the T cell (Evavold et al., 1993, Immunol Today, 14:602-609). In some cases this phenomenon has been related to αβTCR binding kinetics, rather than just overall affinity (Lyons et al., 1996, Immunity, 5:53-61). One could use soluble versions of IA^{b} bound to the peptides identified in this library in surface plasmon resonance studies to address this possibility. Based on these other studies one might predict that those IA^{b}/peptide combinations that stimulated poorly despite their relatively high affinity would turn out to have very fast dissociation rates. The ability to manipulate peptide sequence to produce MHC-mimotope complexes that bind T cells strongly without productive T cell activation could be used to develop tools for the manipulation of T cell responses in vivo.

In general, the present invention has three components: (1) methods for the display of functional MHC molecules with covalently attached antigenic peptides on the surface of baculovirus and baculovirus infected insect cells; (2) methods for the identification and physical isolation of baculovirus or baculovirus infected insect cells bearing a displayed MHC/peptide combination that is recognized by a particular T cell antigen receptor; and (3) methods for producing libraries of baculovirus or baculovirus infected insect cells displaying a particular MHC molecule and many different potential antigenic peptides.

MHC/Peptide Display

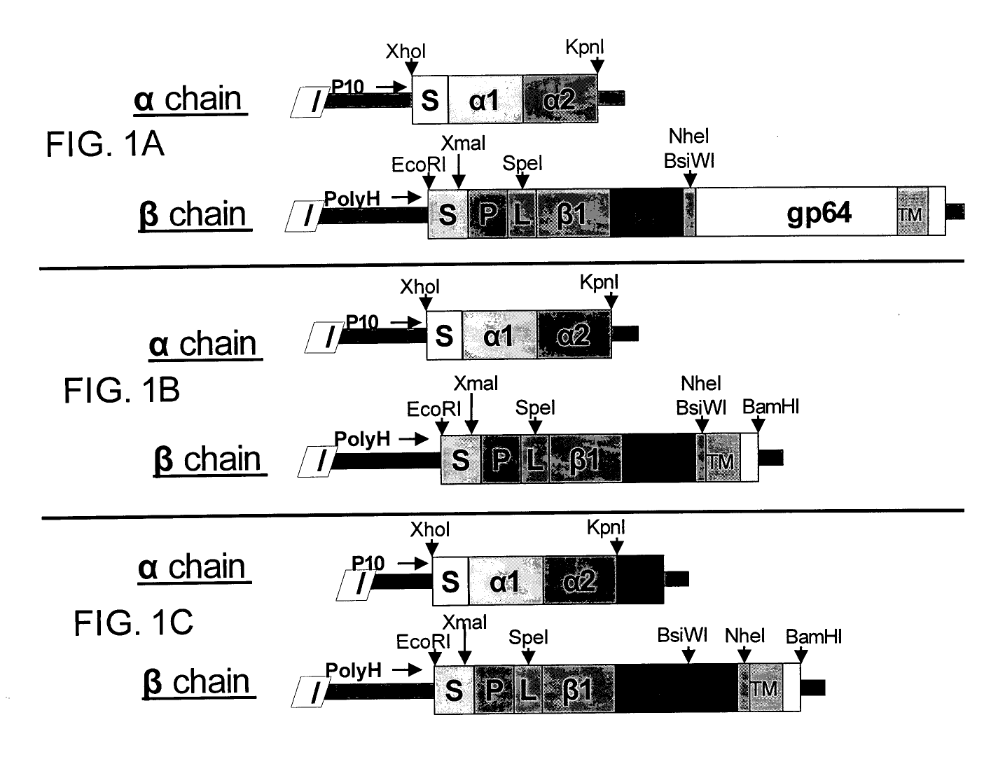

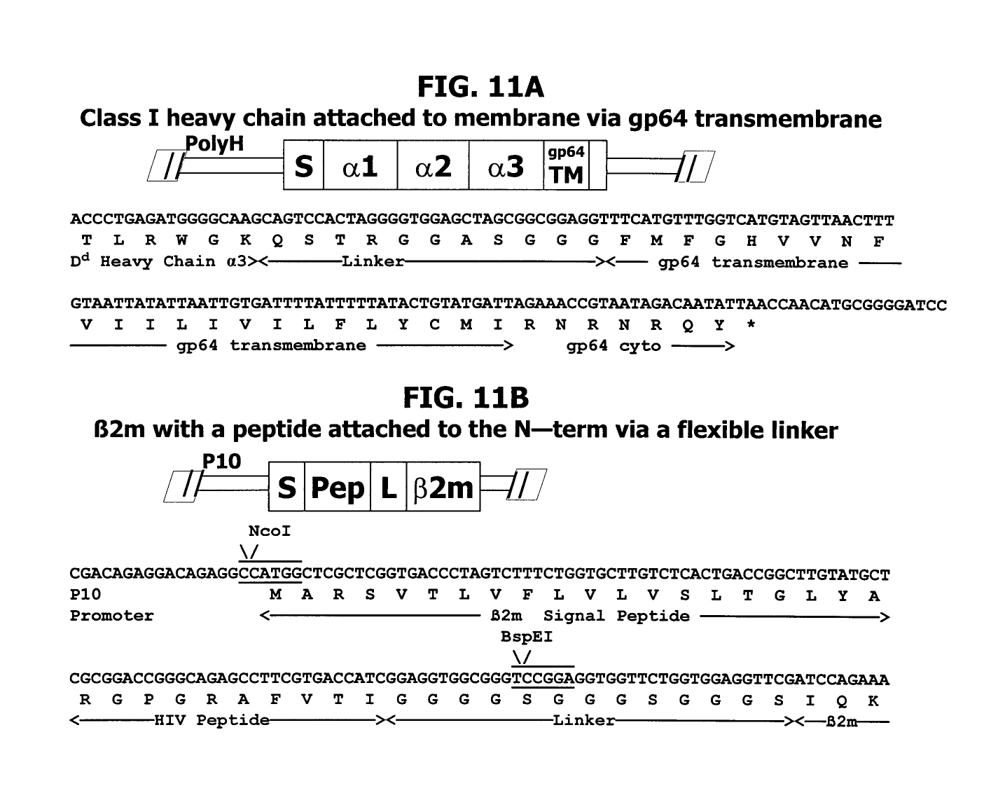

By way of example, but not intended to be limiting to the invention, three different display strategies have been validated by the present inventors using MHC Class II molecules (FIGS. 1A-1C), and one of these has also been validated using MHC Class I molecules (FIG. 11). In all three as used for MHC Class II, a baculovirus was constructed encoding the α and β genes for the MHC molecule. The 3′-ends of the genes were modified to remove sequence encoding the transmembrane region and the cytoplasmic tail. The 5′-end of the β gene was modified to insert a nucleic acid sequence between the signal peptide and the mature β chain encoding an antigenic peptide and a glycine/serine rich linker (Kozono et al., 1994).

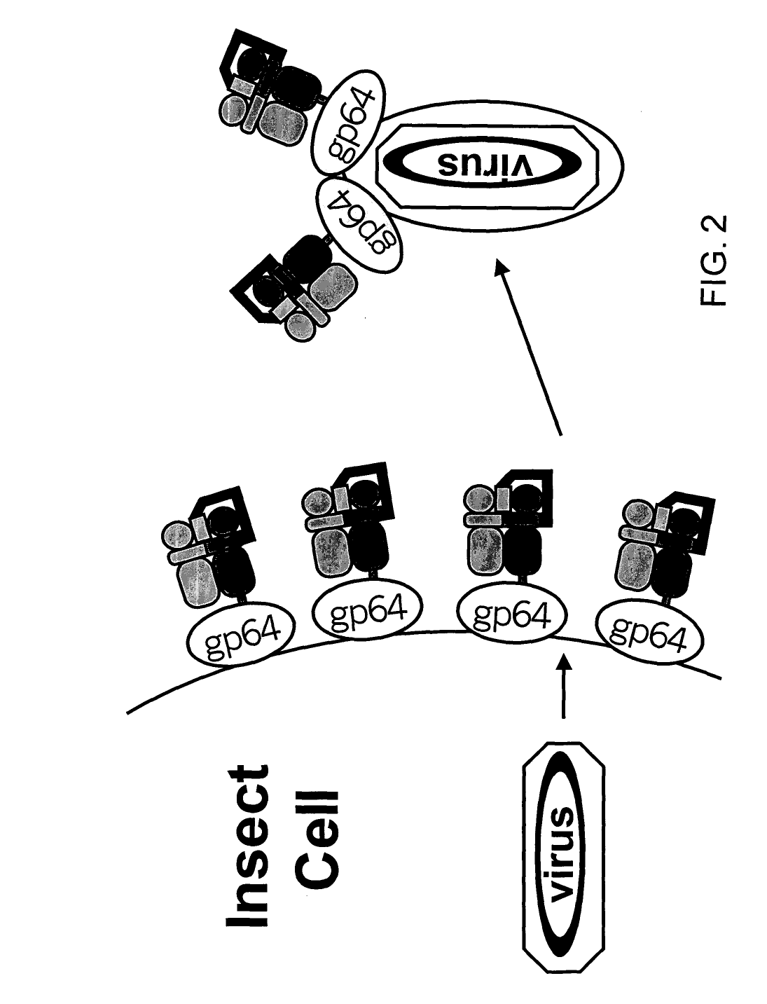

In the first strategy (FIG. 1A), the truncated MHC Class II β gene was also fused in frame with a nucleic acid sequence encoding the full length baculoviral envelop protein, gp64. In the second strategy (FIG. 1B), the MHC Class II β gene was fused to a nucleic acid sequence encoding only the transmembrane and cytoplasmic tail of gp64. This second strategy was also adapted to Class I MHC molecules by fusing the MHC Class I α chain to a nucleic acid sequence encoding only the transmembrane and cytoplasmic tail of gp64, and attaching the antigenic peptide via the β2-microglobulin (β2m) chain used for MHC Class I. In the third strategy (FIG. 1C), the second strategy was expanded by adding a nucleic acid sequence to the end of the α and β genes encoding respectively, basic and acidic leucine zipper dimerization helices (O'Shea et al., 1993). The acidic helix was then attached to the transmembrane and cytoplasmic tail of gp64. In each case, the expression of these constructs in infected insect cells leads to the surface expression of an assembled αβMHC Class II molecule (or in the case of MHC Class I, to the surface expression of an assembled αβ2mMHC Class I molecule) anchored to the insect cell membrane by the β chain via the transmembrane region of gp64 (or by the α chain in MHC Class I constructs). The molecule is fully occupied by the covalently attached antigenic peptide. Normally, baculovirus escapes the infected insect cell by budding through the plasmid membrane, acquiring gp64 on the viral surface in the process. Therefore, with these constructions both the infected insect cells and the virus produced by the cells display the MHC/peptide complex on their surfaces (FIG. 2; MHC Class II diagram).

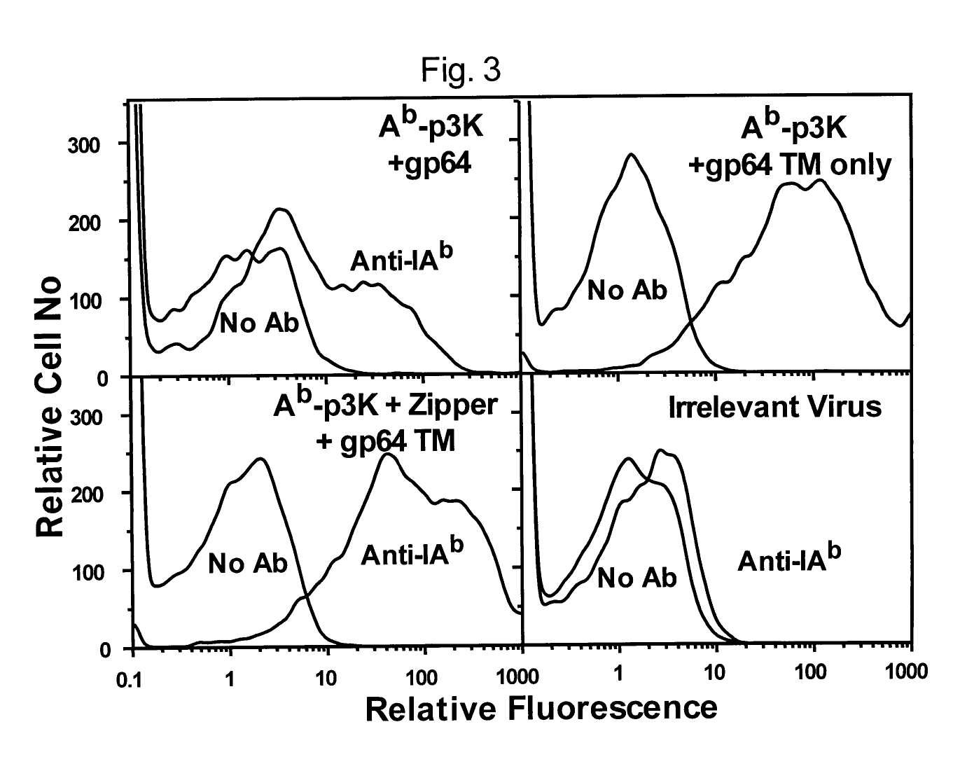

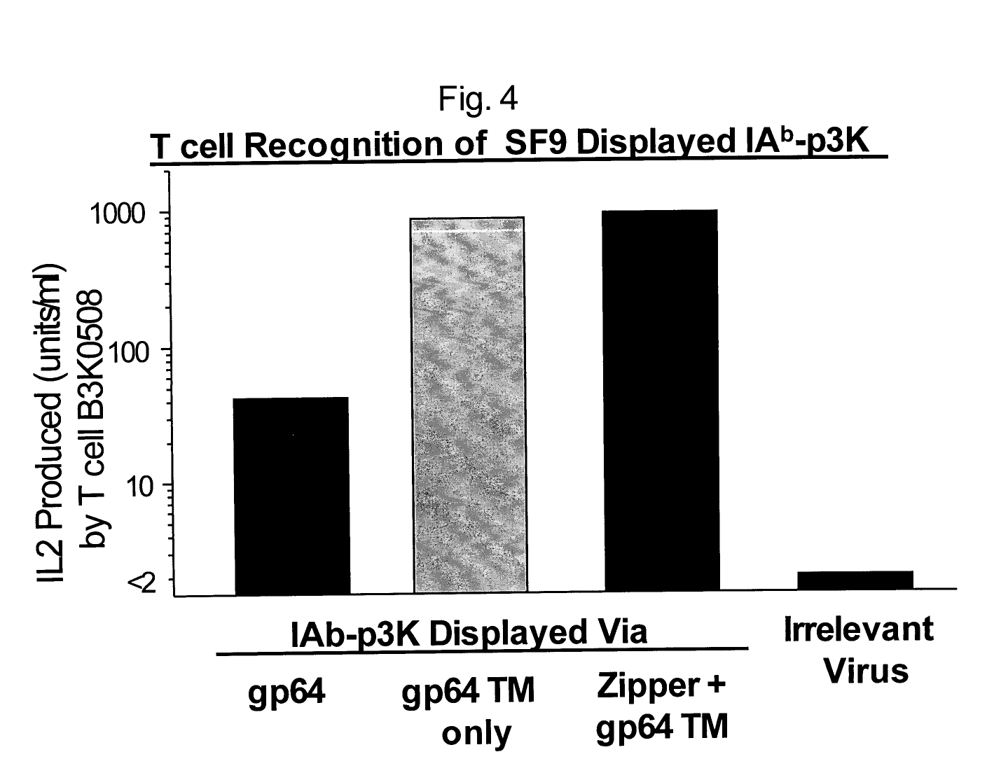

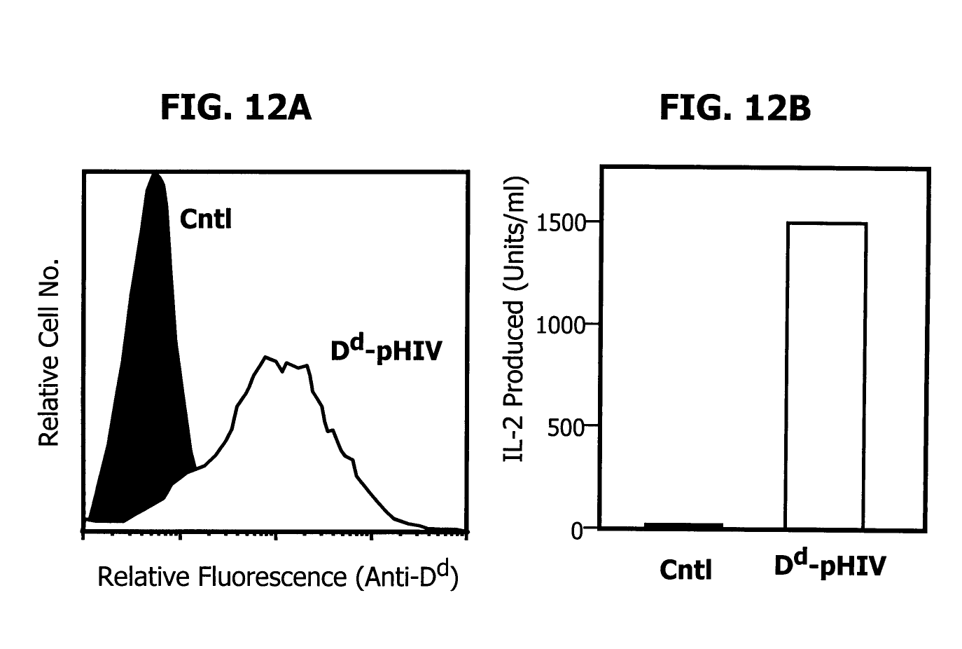

The feasibility of this approach has been confirmed by the inventors with a number of human and mouse MHC Class II molecules carrying covalently attached peptides, as well as with a mouse MHC Class I molecule carrying covalently attached peptides. By way of example, the present inventors produced a functional displayed MHC-peptide complex of the mouse Class II molecule, IA^{b}, and the peptide, p3K, using each of the strategies of the invention (FIG. 3). The functionality of the displayed MHC/peptide complexes in each strategy was shown by the stimulation of T cell hybridomas with receptors of known MHC/peptide specificity (FIG. 4). In another example, the present inventors produced a functional displayed MHC-peptide complex of the mouse Class I molecule, D^{d}, and the peptide pHIV (FIG. 12A). The functionality of the displayed MHC/peptide complex was shown by the stimulation of a T cell with a receptor of known specificity (FIG. 12B) Experiments using the constructs and methods of the invention are described in more detail in the Examples section.

Identification and Isolation of Baculovirus Encoding a Particular MHC/peptide Combination

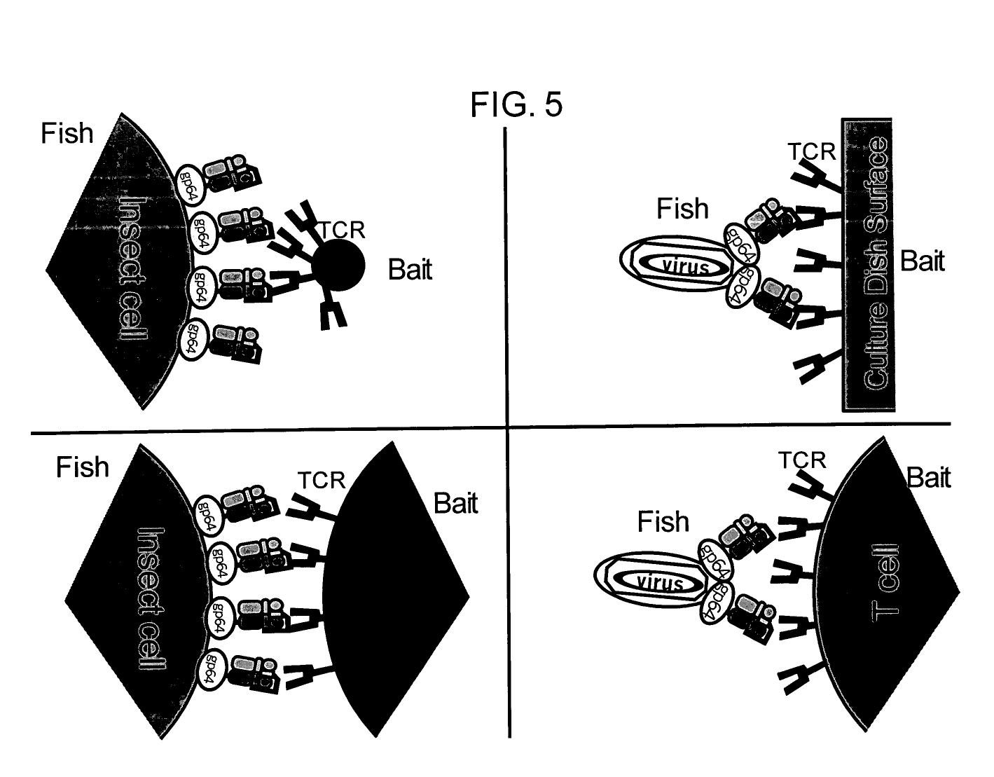

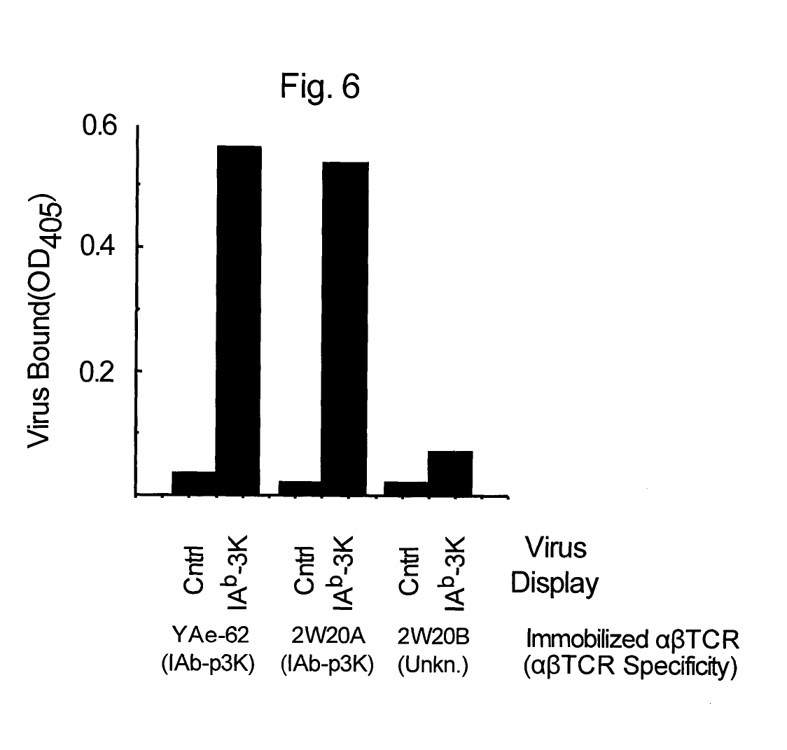

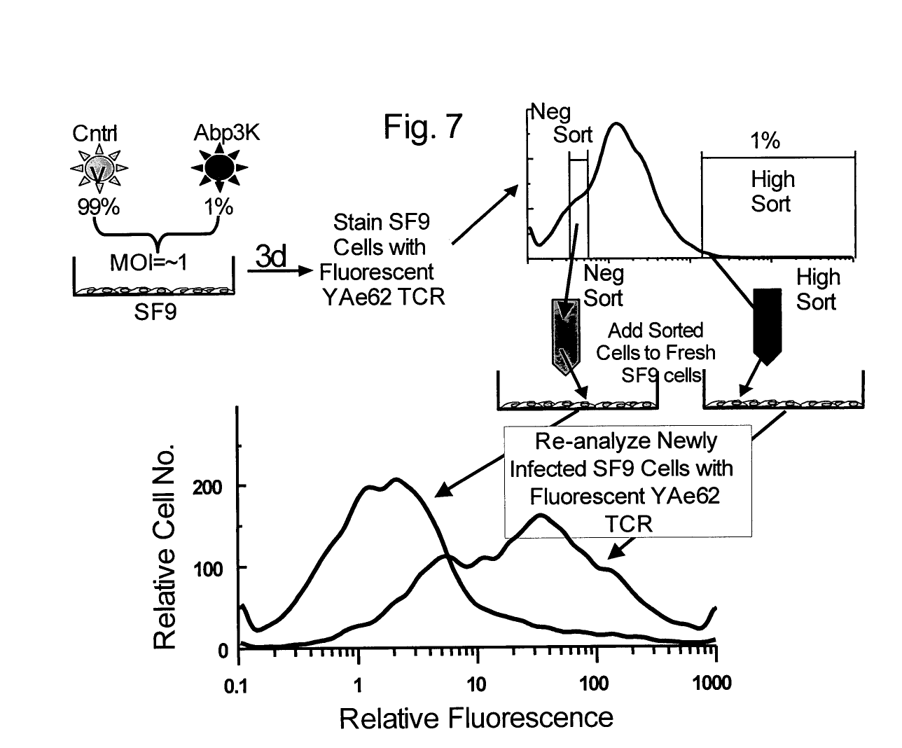

In order to identify and isolate baculoviruses encoding particular MHC/peptide combinations, either T cells or their expressed antigen receptors (i.e., T cell receptors, or TcR) are used as “bait” and baculovirus or baculovirus infected insect cells are used as “fish” (FIG. 5). In the case of baculoviruses, those bearing the appropriate MHC/peptide combinations are bound either to the surface of receptor-bearing T cells or to an immobilized soluble T cell receptor (Kappler et al., 1994). The unbound virus is washed away and the bound virus is used to infect new insect cells for another round of fishing. In the case of baculovirus infected insect cells, fluorescently labeled receptor-bearing T cells or expressed soluble T cell receptor (Kappler et al., 1994) bind to infected insect cells bearing the appropriate MHC/peptide combination. The now fluorescently marked, infected insect cells are identified and separated from non-fluorescent, infected insect cells by flow cytometry and co-cultured with fresh non-infected insect cells to generate new infected cells for another round of fishing. With any of these methods, an enrichment of baculoviruses carrying genes for the correct MHC/peptide combination occurs during each round until eventually viruses carrying other MHC/peptide combinations are lost from the virus stock. At this point, the DNA of individual viruses can be sequenced to determine the peptide sequence. By way of example, FIG. 6 shows the binding of a virus displaying MHC Class II-peptide to an immobilized T cell receptor, and FIG. 7 shows the use of a fluorescently labeled, soluble T cell receptor to bind insect cells displaying MHC-peptide complex.

Construction of Peptide Libraries

In searching for unknown antigenic peptides, the number of different peptides that must be examined depends on the type of experiment and the extent of knowledge about the nature of the peptide/MHC interaction. The core region of antigenic peptides involved in MHC binding and T cell recognition is between about 9-11 amino acids. Therefore, with no other information, a saturating peptide library would require up to 20⁹ or 2×10¹¹ members, which is a very large number and difficult to achieve with any methodology. Fortunately, a fully saturated library is seldom needed. Since 4-5 amino acids are generally involved in MHC binding and can not directly contact the T cell receptor, prior knowledge of the nature of these amino acids means that only about 5-7 amino acids need vary, so that libraries of 10⁶ to 10⁹ members are sufficient. In addition, in some cases T cell recognition is dominated by only a few amino acids in the core of the peptide. In these cases, libraries with only a few hundred to a few thousand members may be sufficient to identify functional peptides.

In order to construct stocks of baculovirus carrying a particular MHC molecule and a library of peptides, the PCR is used to construct a DNA fragment encoding the peptide. By using oligonucleotides that are randomly mutated within particular triplet codons, the resultant fragment pool encodes all possible combinations of codons at these positions. In addition, one can use nucleotide triplets that can be incorporated into oligonucleotides. In this way, codons for each amino acid occur at the same frequency (1:20) and termination codons are eliminated, thus smaller libraries are required. The fragment mixture is then incorporated into baculovirus DNA with the genes encoding the MHC molecule so that each virus encodes the MHC molecule with one version of the peptide covalently attached. The number of viruses that result carrying unique peptides depends on the method of incorporation the DNA fragment, two methods of which are described here, by way of example:

a) Incorporation via Recombination

This method for introducing genes into baculovirus DNA is based on a widely used technique and involves an E. coli plasmid intermediate. The gene is cloned first into an E. coli transfer plasmid where it is flanked by short stretches of baculovirus DNA. The purified plasmid DNA is mixed with baculovirus DNA and transfected into insect cells. Homologous recombination leads to the introduction of the plasmid-encoded gene into the baculovirus DNA and subsequent incorporation into baculovirus. Various commercial modifications of this system lead to the production of only recombinant baculovirus. While very simple to use, recombination frequency in this system is generally low and production of more than 10⁴ to 10⁵ independent viruses is not practical. To modify this method to produce small MHC/peptide libraries according to the present invention, the MHC molecules are encoded in an E. coli transfer plasmid with the region encoding the peptide flanked by two unique restriction enzyme sites. These sites are incorporated into the mutated DNA fragment encoding the peptide during the PCR construction of the fragment. The fragment is then cloned into the transfer plasmid using conventional techniques and a bulk transformation of E. coli is used to produce a mixed population of transfer plasmids, each carrying the MHC genes with sequence for a different peptide attached. The mixture of plasmids is cotransfected with baculovirus DNA into insect cells to produce a mixture of viruses. Even though the original plasmid mixture may encode up to 10⁶ independent peptides, the number that actually end up recombined into baculovirus is generally less than 10⁵.

b) Incorporation via Direct Cloning

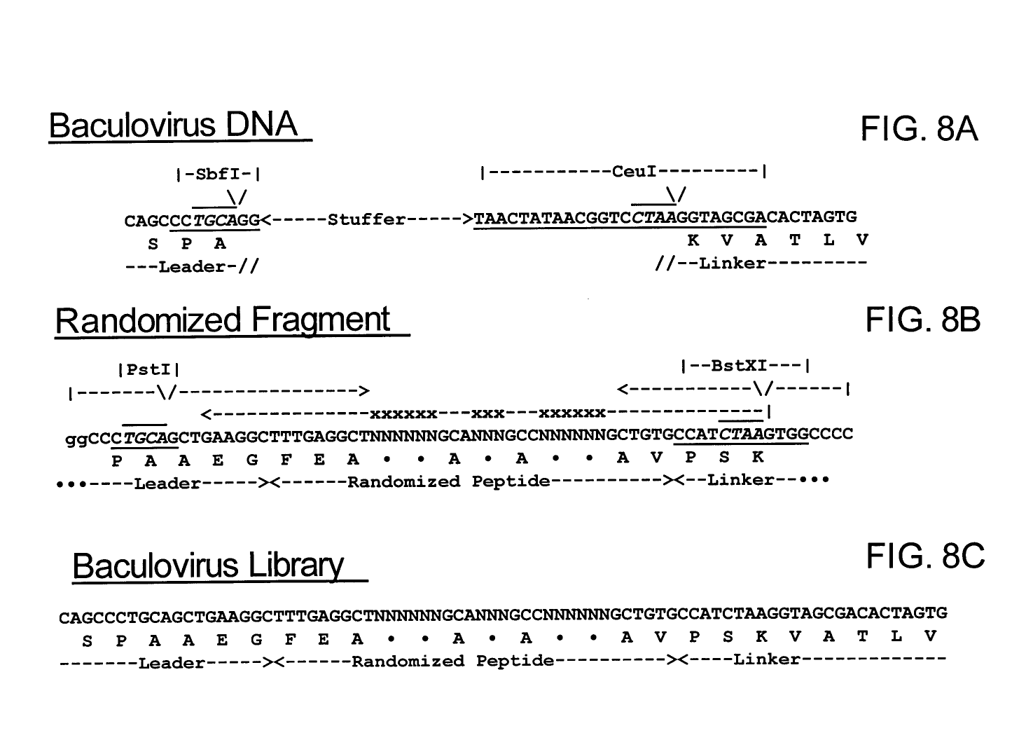

To make larger peptide/MHC libraries, the mutated PCR fragment is cloned directly into baculovirus DNA that already contains the genes for the MHC molecule. This is more difficult than cloning into a transfer plasmid, because the region encoding the peptide must be flanked by sites for unique restriction enzymes that do not cut elsewhere in the baculovirus DNA. Because this DNA is so large (˜135kb), only a few possible enzymes meet this requirement. One pair of enzymes that can be used is SbfI and CeuI. An example of a strategy using these enzymes to construct a IA^{b}/peptide library is described schematically in FIG. 8. It will be apparent that this strategy can be readily applied, using this example, to other MHC molecules and peptides. In this example, baculovirus DNA is constructed containing the IA^{b} genes with sites for these enzymes introduced to flank the peptide site, which is filled with irrelevant stuffer DNA. The stuffer is removed by digestion with SbfI and CeuI. The mutated, peptide-encoding DNA fragment has sites for enzymes that generate compatible ends with SbfI (PstI or NsiI) and CeuI (BstXI). The restricted DNA fragment is then ligated directly into the baculovirus DNA and the ligated DNA is then transfected into insect cells. No recombination is required and each successfully ligated and transfected DNA molecule replicates and yields a unique baculovirus. The number of independent viruses and, therefore, the size of the library, is limited only by the efficiency of ligation. Therefore, libraries of >10⁶ are achievable. There are a number of other restriction enzymes whose recognition sites can be place in a similar manner flanking the peptide site, including but not limited to, SrfI, SceI, AvrI, Bsu36I, PI-pspI, and PI-SceI.

Following are details of the various embodiments of the present invention. One embodiment of the invention relates to a recombinant baculovirus expression vector for expression of functional MHC-peptide molecules. Specifically, the present invention includes a recombinant baculovirus expression vector for expression of functional MHC-peptide molecules that includes a baculovirus genome comprising:

(a) a first nucleic acid sequence inserted into a first baculovirus structural gene at a position under control of a promoter for the first baculovirus structural gene, wherein the first nucleic acid sequence encodes at least a portion of the extracellular domains of the α chain of a major histocompatibility complex (MHC) Class I molecule or at least a portion of the extracellular domains of the α chain of a MHC Class II molecule;

(b) a second nucleic acid sequence inserted into a second baculovirus structural gene at a position under control of a promoter for the second baculovirus structural gene, wherein the second nucleic acid sequence encodes at least a portion of the extracellular domains of either one of:

(i) a β2-microglobulin (β2m) chain of a MHC Class I molecule, if the first nucleic acid sequence encodes at least a portion of the extracellular domains of the α chain of a MHC Class I molecule; or

(ii) a β chain of a MHC Class II molecule, if the first nucleic acid sequence encodes at least a portion of the extracellular domains of the α chain of a MHC Class II molecule;

(c) a third nucleic acid sequence encoding an MHC-binding peptide;

(d) a fourth nucleic acid sequence encoding a peptide linker, wherein the third nucleic acid sequence encoding the MHC-binding peptide is connected to the 5′ end of the first or second nucleic acid sequence by the fourth nucleic acid sequence; and

(e) a fifth nucleic acid sequence encoding at least a transmembrane region of a membrane protein, wherein the first or the second nucleic acid sequence is inserted into the baculovirus genome in frame with the fifth nucleic acid sequence, the fifth nucleic acid sequence being located after the 3′ end of the first or second nucleic acid sequence.

In the baculovirus expression vector of the invention, the portion of the extracellular domains of the α chain of the MHC Class I molecule and the portion of the extracellular domains of the β2m chain of the MHC Class I molecule form a peptide binding groove of an MHC molecule. Similarly, the portion of the extracellular domains of the α chain of the MHC Class II molecule and the portion of the extracellular domains of the β chain of the MHC Class II molecule form a peptide binding groove of an MHC molecule. The MHC-binding peptide comprises a sequence of amino acids that binds to the peptide binding groove.

MHC proteins are generally classified into two categories: class I and class II MHC proteins. An MHC class I protein is an integral membrane protein comprising a glycoprotein heavy chain, also referred to herein as the α chain, which has three extracellular domains (i.e., α₁, α₂ and α₃) and two intracellular domains (i.e., a transmembrane domain (TM) and a cytoplasmic domain (CYT)). The heavy chain is noncovalently associated with a soluble subunit called β2-microglobulin (β2m). An MHC class II protein is a heterodimeric integral membrane protein comprising one α chain and one β chain in noncovalent association. The α chain has two extracellular domains (α₁ and α₂), and two intracellular domains (a TM domain and a CYT domain). The β chain contains two extracellular domains (β₁ and β₂), and two intracellular domains (a TM domain and CYT domain). Many human and other mammalian MHC molecules are well known in the art and any MHC Class I or Class II molecules can be used in the present invention.

According to the present invention, reference to an “MHC-peptide complex” or an “MHC-peptide molecule”, which terms can be used interchangeably, refers to any portion of an MHC protein that forms a functional peptide binding groove and that has a peptide bound to the peptide binding groove. It is well known in the art that the domain organization of class I and class II proteins forms the antigen binding site, or peptide binding groove. A peptide binding groove refers to a portion of an MHC protein that forms a cavity in which a peptide can bind. The conformation of a peptide binding groove is capable of being altered upon binding of an antigenic peptide to enable proper alignment of amino acid residues important for T cell receptor (TcR) binding to the MHC protein and/or peptide. According to the present invention, “a portion” of an MHC chain refers to any portion of an MHC chain that is sufficient to form a peptide binding groove upon association with a sufficient portion of another chain of an MHC protein. In one embodiment, portions of MHC chains suitable to form a peptide binding groove are the portions of MHC chains that are suitable to produce a soluble MHC protein, and particularly include any suitable portion of the extracellular domains of an MHC chain. A soluble MHC protein lacks amino acid sequences capable of anchoring the molecule into a lipid-containing substrate, such as an MHC transmembrane domain and/or an MHC cytoplasmic domain.

For example, a peptide binding groove of a class I protein can comprise portions of the α₁, and α₂ domains of the heavy chain capable of forming two β-pleated sheets and two α helices. Inclusion of a portion of the β2-microglobulin chain stabilizes the complex. While for most versions of MHC Class II molecules, interaction of the α and β chains can occur in the absence of a peptide, the two chain complex of MHC Class I is unstable until the binding groove is filled with a peptide.

A peptide binding groove of a class II protein can comprise portions of the α₁ and β₁ domains capable of forming two β-pleated sheets and two α helices. A first portion of the α₁ domain forms a first β-pleated sheet and a second portion of the α₁ domain forms a first α helix. A first portion of the β₁ domain forms a second β-pleated sheet and a second portion of the β₁ domain forms a second α helix. The X-ray crystallographic structure of class II protein with a peptide engaged in the binding groove of the protein shows that one or both ends of the engaged peptide can project beyond the MHC protein (Brown et al., pp. 33-39, 1993, Nature, Vol. 364). Thus, the ends of the α₁ and β₁ α helices of class II form an open cavity such that the ends of the peptide bound to the binding groove are not buried in the cavity. Moreover, the X-ray crystallographic structure of class II proteins shows that the N-terminal end of the MHC β chain apparently projects from the side of the MHC protein in an unstructured manner since the first 4 amino acid residues of the β chain could not be assigned by X-ray crystallography.

An MHC-binding peptide (e.g., an antigenic peptide or T cell epitope) of the present invention can comprise any peptide that is capable of binding to an MHC protein in a manner such that the MHC-peptide complex can bind to a T cell receptor (TcR) and, in a preferred embodiment, thereby induce a T cell response. An MHC-binding peptide that binds to an MHC molecule and is recognized, in conjunction with the MHC molecule, by a T cell receptor, is considered to be an antigenic peptide. As such, a “candidate antigenic peptide” and an “MHC-binding peptide” can be used interchangeably, when the MHC-binding peptide is produced to be a candidate for T cell receptor binding. Since many MHC-binding peptides of the present invention are only candidates for T cell receptor recognition, an MHC-binding peptide is not necessarily an antigenic peptide, even though it may be included in a given recombinant baculovirus according to the present invention. Indeed, in large peptide libraries where less is known about the requirements for MHC binding of peptides (and thus the design of the peptide is more random), some peptides may not bind the MHC peptide binding groove at all or only minimally when the recombinant vector is expressed. Such MHC molecules will not be stable and will not be selected for binding to a T cell receptor, and in many cases, if no peptide binds to the MHC peptide binding groove, the complex may denature in the endoplasmic reticulum and not be expressed at all by the baculovirus. Examples of MHC-binding peptides can include peptides produced by hydrolysis and most typically, synthetically produced peptides, including randomly generated peptides, specifically designed peptides, and peptides where at least some of the amino acid positions are conserved among several peptides and the remaining positions are random.

In nature, peptides that are produced by hydrolysis of antigens undergo hydrolysis prior to binding of the antigen to an MHC protein. Class I MHC proteins typically present antigenic peptides derived from proteins actively synthesized in the cytoplasm of the cell. In contrast, class II MHC proteins typically present antigenic peptides derived either from exogenous proteins that enter a cell's endocytic pathway or from proteins synthesized in the ER. Intracellular trafficking permits an antigenic peptide to become associated with an MHC protein. The resulting MHC-peptide complex then travels to the surface of the cell where it is available for interaction with a TcR.

The binding of a peptide to an MHC peptide binding groove can control the spatial arrangement of MHC and/or peptide amino acid residues recognized by a TcR. Such spatial control is due in part to hydrogen bonds formed between a peptide and an MHC protein. As discussed above with regard to IA^{b}, enough is known about how peptides bind to various MHC molecules to determine what are the major MHC anchor amino acids of a peptide which are typically held constant, and what are the surface exposed amino acids that are varied among different peptides. Preferably, the length of an MHC-binding peptide is from about 5 to about 40 amino acid residues, more preferably from about 6 to about 30 amino acid residues, and even more preferably from about 8 to about 20 amino acid residues, and even more preferably between about 9 and 11 amino acid residues, including any size peptide between 5 and 40 amino acids in length, in whole integer increments (i.e., 5, 6, 7, 8, 9 . . . 40). While naturally MHC Class II-bound peptides vary from about 9-40 amino acids, in nearly all cases the peptide can be truncated to an about 9-11 amino acid core without loss of MHC binding activity or T cell recognition.

Peptides used in the invention can include peptides comprising at least a portion of an antigen selected from a group consisting of autoantigens, infectious agents, toxins, allergens, or mixtures thereof. However, a main aspect of the invention is the use of synthetically produced peptides to identify the antigens recognized by a specific T cell. Therefore, preferred peptides are from libraries of synthetically produced peptides, including, but not limited to, peptide libraries produced by PCR (including by introducing random mutations into various positions of a template peptide). As discussed above, a peptide library can include up to 20⁹ or 2×10¹¹ members, or as few as a few hundred to a few thousand members, depending on the knowledge of the peptide binding characteristics of a given MHC molecule. Since 4-5 amino acids are generally involved in MHC binding and can not directly contact the T cell receptor, prior knowledge of the nature of these amino acids means that only about 5-7 amino acids in the peptide need vary, so that libraries of 10⁶ to 10⁹members are typically sufficient. In addition, in some cases, T cell recognition is dominated by only a few amino acids in the core of the peptide, and in these cases, libraries with only a few hundred to a few thousand members may be sufficient to identify functional peptide-MHC complexes.

Extensive knowledge regarding the binding of peptides to MHC complexes is available to the public, so that for a given MHC complex, one can design MHC-groove binding peptides that vary in less than all of the available positions. For example, the MHCBN is a comprehensive database of Major Histocompatibility Complex (MHC) binding and non-binding peptides compiled from published literature and existing databases. The latest version of the database has 19,777 entries including 17,129 MHC binders and 2648 MHC non-binders for more than 400 MHC molecules. The database has sequence and structure data of (a) source proteins of peptides and (b) MHC molecules. MHCBN has a number of web tools that include: (i) mapping of peptide on query sequence; (ii) search on any field; (iii) creation of data sets; and (iv) online data submission (Bioinformatics 2003 Mar. 22;19(5):665-6).

In one embodiment of the invention, the MHC-binding peptide is from a library of candidate antigenic peptides, wherein the each of the peptides in the library comprises conserved amino acids in a specific sequence sufficient to enable the peptide to bind to the peptide binding groove of the MHC molecule that is encoded by the vector. In a more specific embodiment, the MHC-binding peptide is from a library of candidate antigenic peptides, wherein each of the peptides in the library comprises between about 4 and 5 conserved amino acids in a specific sequence sufficient to enable the peptide to bind to the peptide binding groove of the MHC molecule that is encoded by the vector. In another embodiment, the MHC-binding peptide is from a library of candidate antigenic peptides representing from between about 10³ and about 10⁹ different candidate antigenic peptides.

In a preferred embodiment, a library of candidate peptides (candidate antigenic peptides or MHC-binding peptides) is produced by genetically engineering the library using polymerase chain reaction (PCR) or any other suitable technique to construct a DNA fragment encoding the peptide. With PCR techniques, by using oligonucleotides that are randomly mutated within particular triplet codons, the resultant fragment pool encodes all possible combination of codons at these positions. Preferably, certain of the amino acid positions are maintained constant, which are the conserved amino acids that are required for binding to the MHC peptide binding groove and which do not contact the T cell receptor.

The fourth nucleic acid sequence in the expression vector of the invention encodes a peptide linker, wherein the third nucleic acid sequence encoding the MHC-binding peptide is connected to the 5′ end of the first or second nucleic acid sequence (encoding α chain of the MHC molecule) by the fourth nucleic acid sequence encoding the linker (i.e., the linker is located between the MHC molecule portion and the MHC-binding peptide). When translated into a protein, the peptide linker therefore covalently links the MHC-binding peptide to one of the MHC portions. By producing the complex recombinantly, covalent bonds are formed between the MHC-binding peptide and the peptide linker, and between the linker and the MHC segment. The peptide linker is distinguished from a peptide linkage which refers to the chemical interaction between two amino acids. In one embodiment, when the MHC part of the complex is a Class I molecule, the third nucleic acid sequence encoding the MHC-binding peptide is connected to the 5′ end of the second nucleic acid sequence encoding at least a portion of the extracellular domains of a β2m chain of a MHC Class I molecule by the fourth nucleic acid sequence encoding a peptide linker. In another embodiment, when the MHC part of the complex is a Class II molecule, the third nucleic acid sequence encoding the MHC-binding peptide is connected to the 5′ end of the second nucleic acid sequence encoding at least a portion of the extracellular domains of a β chain of a MHC Class II molecule by the fourth nucleic acid sequence encoding a peptide linker. It is not required that the peptide linker and MHC-binding peptide be attached to the β2m chain of the Class I molecule or to the β chain of the Class II molecule, as attachment to the α chains of either MHC molecule can also be achieved.

A peptide linker encoded by a nucleic acid sequence useful in recombinant expression vector of the invention can comprise any amino acid sequence that facilitates the binding of a peptide to a peptide binding groove of an MHC molecule. For example, a peptide linker can facilitate peptide binding by, for example, maintaining the peptide within a certain distance of an MHC peptide binding groove to promote efficient binding. The peptide linker of the present invention also stabilizes the association of an MHC-binding peptide with an MHC peptide binding groove, resulting in the formation of a stable complex that can be recognized by a TCR. As used herein, the term “stability” refers to the maintenance of the association of a peptide with an MHC peptide binding groove in the presence of forces that could typically cause the dissociation of complexed peptide and MHC protein. The stability of a peptide bound to an MHC peptide binding groove can be measured in a variety of ways known to those skilled in the art, including by high pressure liquid chromatography (HPLC), or by incubating in increasing concentrations of sodium dodecyl sulfate (SDS) for an appropriate amount of time and at an appropriate temperature. The stability of the MHC-peptide complexes formed by the method of the present invention preferably is substantially the same as or greater than the stability of a native form of the complex.

Furthermore, a peptide linker used in the complex of the invention can include an amino acid sequence that does not substantially hinder interaction of an MHC-binding peptide with an MHC peptide binding groove or the interaction of an MHC-peptide complex with a TcR. For example, the length of a peptide linker of the present invention is preferably sufficiently short (i.e., small enough in size) such that the linker does not substantially inhibit the binding between the MHC-binding peptide and the MHC peptide binding groove or inhibit TCR recognition. Preferably, the length of a linker of the present invention is from about 1 amino acid residue to about 40 amino acid residues, more preferably from about 5 amino acid residues to about 30 amino acid residues, and even more preferably from about 8 amino acid residues to about 20 amino acid residues, including any length peptide between 1 and about 40 amino acid residues, in whole integer increments (i.e., 1, 2, 3, 4, 5, 6, . . . 40). In one embodiment, the peptide linker is at least about 5 amino acids in length, or at least about 6 amino acids in length, or at least about 7 amino acids in length, or at least about 8 amino acids in length, and so on, in whole integer increments, up to about 40 amino acids in length. Longer peptide linkers could also be used, as long as the linker does not hinder the MHC-peptide interactions as discussed above. Most typically, a peptide linker is between about 15-16 amino acids in length, counting from amino acid position 9 of the MHC Class II peptide or from the C-term of the MHC Class I peptide, to about amino acid position 4 of MHC Class II β chain, or to the N-terminus of β2m, respectively. This is an example of an optimum length to link the MHC to the peptide without conflict, and not disrupt TCR recognition.

The peptide linker of the present invention is preferably substantially neutral such that the linker does not inhibit MHC-peptide complex formation or TCR recognition of the complex. As used herein, the term “neutral” refers to amino acid residues sufficiently uncharged or small in size so that they do not prevent interaction of a peptide with an MHC molecule (e.g., with the peptide binding groove). Preferred amino acid residues for peptide linkers of the present invention include, but are not limited to glycine, alanine, leucine, serine, valine, threonine, and proline residues. More preferred linker amino acid residues include glycine, serine, leucine, valine, and proline residues. Linker compositions can also be interspersed with additional amino acid residues, such as arginine residues. Linker amino acid residues of the present invention can occur in any sequential order such that there is no interference with binding of an MHC-binding peptide to the MHC molecule or of the resulting MHC-peptide complex with a TCR. Such peptide linkers and methods of identifying and producing such linkers have been described in detail in U.S. Pat. No. 5,820,866, issued Oct. 13, 1998, which is incorporated herein by reference in its entirety.

A fifth nucleic acid sequence in the recombinant expression vector of the present invention encodes at least a transmembrane region of a membrane protein, wherein the first or the second nucleic acid sequence is inserted into the baculovirus genome in frame with the fifth nucleic acid sequence, the fifth nucleic acid sequence being located after the 3′ end of the first or second nucleic acid sequence. The purpose of this portion of the complex is to achieve the surface expression of an assembled MHC-peptide complex that is anchored to baculovirus membrane or to the insect cell membrane via the transmembrane region of the protein encoded by the fifth nucleic acid sequence. As discussed above, baculovirus normally escapes the infected insect host cell by budding through the plasmid membrane, and acquiring gp64 on the viral surface in the process. gp64 is baculoviral envelop protein and therefore, the use of at least the transmembrane region of this protein is suitable for the present invention, as expression vectors encoding at least the gp64 transmembrane protein will cause the display of the MHC-peptide complex on the surface of both the baculovirus and the infected host cell. In one aspect of the invention, the fifth nucleic acid sequence encodes a full-length gp64 protein, the transmembrane and cytoplasmic portions of gp64, or a protein comprising just the transmembrane region of gp64.

The invention is not limited to the use of the gp64 transmembrane region or proteins comprising this region of gp64, as many other transmembrane regions of membrane proteins could be used to achieve the same effect. For example, the method could be adapted to Class I MHC molecules by anchoring the molecule via the heavy chain and attaching the antigenic peptide via the β2-microglobulin (β2m) chain (White et al., 1999). In other embodiments, transmembrane regions from other membrane proteins (including larger proteins comprising such regions) can be encoded by the fifth nucleic acid molecule. Such membrane proteins include, but are not limited, such as MHC Class I or II, and other envelope proteins, such as p26.

In one aspect of the invention, the first nucleic acid sequence further comprises, 3′ of the nucleic acid sequence encoding the extracellular domains of the α chain of an MHC molecule, a nucleic acid sequence encoding a basic leucine zipper dimerization helix. In another embodiment, the second nucleic acid sequence comprises, 3′ of the nucleic acid sequence encoding the extracellular domains of the β chain of a Class II MHC molecule or the Class I β2m molecule, a nucleic acid sequence encoding an acidic leucine zipper dimerization helix. The nucleic acid sequence encoding the acidic helix is then attached to the nucleic acid sequence encoding the transmembrane region of a membrane protein. In one embodiment, both the basic leucine zipper dimerization helix and the acidic leucine zipper dimerization helix can be included in the vector, attached to the MHC chains as described above. The result of adding this sequence is that surface expression of an assembled MHC molecule anchored to the insect cell membrane by the chain containing the transmembrane region of the membrane protein is readily achieved.

It will be apparent from the discussion above that the third, fourth and fifth nucleic acid sequences of the expression vector of the invention are incorporated into the baculovirus genome in frame with and either directly attached to or proximal to (e.g., separated by no more than about 1 to about 500 bp), either the first or second nucleic acid sequence of the vector, depending on how the vector is to be constructed. For example, the third nucleic acid sequence encoding the MHC-binding peptide is directly attached to the fourth nucleic acid sequence encoding the peptide linker which is in turn directly attached to the 5′ end of either the first or second nucleic acid sequence, depending on whether the peptide is to be attached to the α chain of the MHC molecule (Class I or Class II), or to the β chain (Class II) or β2m chain (Class I). The fifth nucleic acid sequence encoding the transmembrane protein is placed after the 3′ end of the first or second nucleic acid sequence and in frame with that sequence (and either directly attached to the sequence or separated by a small number of bp (e.g., between 1 and 500 bp that effectively encode a peptide linker).

Attaching the peptide to the MHC Class I or MHC Class II molecule via a flexible linker has the advantage of assuring that the peptide will occupy and stay associated with the MHC molecule during biosynthesis, transport and display. However, there may be situations in which this linker interferes with peptide binding to the MHC molecule or with αβTCR recognition of the complex. As an alternate approach, in one embodiment of the present invention, the MHC molecule and the peptide library are expressed separately in the insect cell. In this case, the MHC chains, in the absence of the linked MHC-binding peptide, would be cloned into a conventional expression vector that has been modified by the present inventors and that uses insect promoters and enhancers. These constructs are transfected directly into insect cells to produce a permanently transfected cell line that expresses both MHC chains, but no peptide. The present inventors have prepared an efficient insect cell expression vector based on the baculovirus IE1 promoter and hr5 enhancer. This vector system can be used to stably express a displayable MHC Class I or MHC Class II molecule in an insect cell, but in this case without a covalently attached peptide. This method has been used by the inventors successfully to produce proteins in insect cells including GFP, B7 and ICAM (see Example 1). Briefly, and merely by way of example, as other promoter/enhancer combinations can be used if desired, DNA fragments encoding the baculovirus hr5 enhancer element, IE1 gene promoter, and IE1 polyA addition region were synthesized by PCR using baculovirus DNA as a template. The fragments were used to construct an insect cell expression vector (PTIE1) on a pTZ18R (Pharmacia) backbone with the hr5 enhancer at the 5′ end, followed by the IE1 promoter, a large multiple cloning site (Esp3I, MunI, SalI, XhoI, BsrGI, HpaI, SpeI, BstXI, BamHI, BspEI, NotI, SacII, XbaI) and the IE1 polyA addition region. DNA fragments encoding the desired protein are cloned into the multiple cloning site and insect cells are transfected with the plasmids using conventional techniques.

In this embodiment of the invention, the insect cells that have been transfected with the plasmids encoding the MHC chains are then infected with baculovirus carrying the unlinked peptide library. The peptide library can be constructed in baculovirus as before, without an attached MHC molecule, but still with an N-terminal attached signal sequence to direct the peptide into the endoplasmic reticulum. The signal peptide is cleaved off naturally, leaving the free peptide to bind to the MHC Class I or Class II molecule produced by the insect cell to complete the MHC-peptide complex for display on the insect cell surface. The strength of the baculovirus polyhedrin promoter is expected to lead to over-expression of the peptide in considerable molar excess over the MHC molecule. One can expect loading of the peptide during MHC biosynthesis and folding followed by transport to the cell surface. At this point the methodology of library screening and manipulation will be as before.

Therefore, one embodiment of the present invention relates to a recombinant insect cell that displays MHC-peptide complexes, including MHC-peptide libraries, on its surface. The recombinant insect cell is transfected with recombinant nucleic acid molecules that encode at least the extracellular domains of an MHC molecule. The recombinant nucleic acid molecules include: (a) a first nucleic acid sequence operatively linked to an expression control sequence, wherein the first nucleic acid sequence encodes at least a portion of the extracellular domains of the α chain of a major histocompatibility complex (MHC) Class I molecule or at least a portion of the extracellular domains of the α chain of a MHC Class II molecule; and (b) a second nucleic acid sequence operatively linked to an expression control sequence under control of a baculovirus promoter and enhancer, wherein the second nucleic acid sequence encodes at least a portion of the extracellular domains of: (1) a β2-microglobulin (β2m) chain of a MHC Class I molecule if the first nucleic acid sequence encodes at least a portion of the extracellular domains of the α chain of a MHC Class I molecule; or (2) a β chain of a MHC Class II molecule if the first nucleic acid sequence encodes at least a portion of the extracellular domains of the α chain of a MHC Class II molecule. The portion of the extracellular domains of the α chain of the MHC Class I molecule and the portion of the extracellular domains of the β2m chain of the MHC Class I molecule, or the portion of the extracellular domains of the α chain of the MHC Class II molecule and the portion of the extracellular domains of the β chain of the MHC Class II molecule, form a peptide binding groove of an MHC molecule. The MHC chain constructs can be transfected into the insect cell in a single recombinant nucleic acid molecule or in different recombinant nucleic acid molecules. The transfected recombinant insect cell is then transfected with recombinant baculoviruses comprising a third nucleic acid sequence under control of a baculovirus promoter and comprising a signal sequence. The third nucleic acid sequence encodes an MHC-binding peptide, wherein the MHC-binding peptide comprises a sequence of amino acids that binds to the peptide binding groove of the MHC Class I molecule or the MHC Class II molecule. The baculoviruses can comprise the peptide libraries as described previously herein. Upon infection, as discussed above, the peptides are produced in the cell and complex with the MHC molecules produced by the insect cell. The resulting complex is displayed on the insect cell surface and the various screening methods described herein can be performed as described. It is to be understood that this approach can be substituted into any of the methods discussed herein for the screening of peptides and peptide libraries.

Production of recombinant constructs (e.g., recombinant nucleic acid molecules) comprising combinations of the first or second, and third, fourth and/or fifth nucleic acid sequences of the invention (or which encode just the peptide library with signal sequence as described for the alternate embodiment above), which are then introduced into the baculovirus genome are known in the art. Methods for producing a recombinant nucleic acid molecule encoding a portion of an MHC molecule covalently attached to a peptide linker and MHC-binding peptide are described in detail in U.S. Pat. No. 5,820,866, supra.

In general, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences (e.g., the first, second, third, fourth or fifth sequence to be included in the recombinant baculovirus, which is also a recombinant vector) and can include nucleic acid sequences that are not naturally found adjacent to nucleic acid sequences of choice (e.g., promoters, untranslated regions). The phrase “recombinant nucleic acid molecule” is used primarily to refer to a recombinant vector into which has been ligated the nucleic acid sequence to be cloned, manipulated, transformed into the host cell (i.e., the insert). “DNA construct” can be used interchangeably with “recombinant nucleic acid molecule” in some embodiments and is further defined herein to be a constructed (non-naturally occurring) DNA molecules useful for introducing DNA into host cells, and the term includes chimeric genes, expression cassettes, and vectors.

In one embodiment, a recombinant vector of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector (e.g., a baculovirus vector) to produce a recombinant nucleic acid molecule. The nucleic acid sequence encoding the protein to be produced is inserted into the vector in a manner that operatively links the nucleic acid sequence to regulatory sequences in the vector (e.g., a promoter) which enable the transcription and translation of the nucleic acid sequence within the recombinant host cell (e.g., an insect cell).

According to the present invention, the phrase “operatively linked” refers to linking a nucleic acid molecule to an expression control sequence in a manner such that proteins encoded by the nucleic acid sequence can be expressed when transfected (i.e., transformed, transduced, transfected, conjugated or conduced) into a host cell. Methods of operatively linking expression control sequences to coding sequences are well known in the art. See, e.g., Maniatis et al., Molecular Cloning: A Laboratory Manual, Cold Spring Harbor, NY (1982), Sambrook et al., Molecular Cloning: A Laboratory Manual, Cold Spring Harbor, NY (1989). Expression control sequences can include sequences that control transcription and/or translation. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell useful in the present invention. The transcription control sequences includes a promoter. The promoter may be any DNA sequence which shows transcriptional activity in the chosen host cell or organism. As discussed above, when the nucleic acid sequences of the invention are ultimately cloned into a recombinant baculovirus genome, the sequences will be introduced into a structural gene under the control of a baculovirus promoter. In manipulating recombinant constructs prior to introduction of the construct into the baculovirus, any suitable promoter can be used depending on the recombinant vector and host cell used. Recombinant nucleic acid molecules of the present invention can also contain additional regulatory sequences, such as translation regulatory sequences, origins of replication, and other regulatory sequences that are compatible with the recombinant cell.

It will be appreciated by one skilled in the art that use of recombinant DNA technologies can improve control of expression of transformed nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter.

The first and second nucleic acid sequences and the associated third, fourth or fifth nucleic acid sequences, are inserted into the baculovirus genome at a position under control of promoters for a first and second baculovirus structural gene, respectively, which causes the first though fifth nucleic acid sequences to be expressed when the baculovirus infects a suitable host cell. The baculovirus genome is well known (Ayres, M et al. Virology 202: 586 (1994)) and therefore, it is well within the ability of one of skill in the art to produce the recombinant baculovirus expression vector according to the invention, given the guidance provided herein. The constructs can be prepared and introduced into the baculovirus by any suitable technique, but two particularly preferred methods are use of an E. coli transfer plasmid, or by direct cloning of the sequences into the genome. Each of these techniques has been discussed in detail above with regard to the present invention. Molecular techniques required to perform such methods for genetic manipulation of the baculovirus genome are well known in the art and are described, for example, in Sambrook et al., Molecular Cloning: A Laboratory Manual, Cold Spring Harbor Labs Press, 1989. In addition, methods for genetic manipulation of the baculovirus genome and production of recombinant baculoviruses are described with regard to the present invention in the Examples and above, and in general in Baculovirus Expression Vectors: A Laboratory Manual, O'Reilly, D. et al. Oxford University Press (1994).

Another embodiment of the present invention relates to a method to produce libraries of functional MHC-peptide molecules displayed on the surface of baculovirus and baculovirus-infected cells. More specifically, the method includes production of libraries of functional MHC-peptide molecules displayed on the surface of baculovirus and baculovirus-infected cells, comprising the steps of: (a) producing a population of recombinant baculoviruses as previously described herein (and discussed in more detail below); and (b) expressing the nucleic acid sequences encoded by the recombinant baculoviruses on the surface of each of the baculoviruses in the population, wherein expression of the nucleic acid sequences results in the production of at least a portion of an MHC molecule which is covalently linked to a candidate antigenic peptide expressed by the given baculovirus via the peptide linker, and wherein the candidate antigenic peptide is bound to the peptide binding groove of the MHC molecule, thereby forming a library of MHC-peptide molecules displayed on the surface of baculoviruses, the library representing multiple different candidate antigenic peptides.

In this embodiment, the population of recombinant baculoviruses is produced by introducing into the genome of the baculoviruses:

(i) a first nucleic acid sequence encoding at least a portion of the extracellular domains of the α chain of a major histocompatibility complex (MHC) Class I molecule or at least a portion of the extracellular domains of the α chain of a MHC Class II molecule, wherein the first nucleic acid sequence is introduced into the baculovirus genome at a position under control of a promoter for a first baculovirus structural gene;

(ii) a second nucleic acid sequence encoding at least a portion of the extracellular domains of:

(1) a β2-microglobulin (β2m) chain of a MHC Class I molecule if the first nucleic acid sequence encodes at least a portion of the extracellular domains of the α chain of a MHC Class I molecule; or

(2) a β chain of a MHC Class II molecule if the first nucleic acid sequence encodes at least a portion of the extracellular domains of the α chain of a MHC Class II molecule;

wherein the second nucleic acid sequence is introduced into the baculovirus genome at a position under control of a promoter for a second baculovirus structural gene; and

wherein the portion of the extracellular domains of the α chain of the MHC Class II molecule and the portion of the extracellular domains of the β chain of the Class II MHC molecule, or the portion of the extracellular domains of the α chain of the Class I MHC molecule and the portion of the extracellular domains of the β2m chain of the Class I MHC molecule, respectively, form a peptide binding groove;

(iii) a third nucleic acid sequence encoding a candidate antigenic peptide, wherein the third nucleic acid sequence is introduced into the baculovirus genome before the 5′ end of the first or second nucleic acid sequence;

(iv) a fourth nucleic acid sequence encoding a peptide linker, wherein the third nucleic acid sequence encoding a candidate antigenic peptide is connected to the first or second nucleic acid sequence by the fourth nucleic acid sequence; and

(v) a fifth nucleic acid sequence encoding at least the transmembrane portion of a membrane protein, the membrane protein-encoding sequence being in frame with and located after the 3′ end of the first or second nucleic acid sequence.

In this embodiment, the candidate antigenic peptide (equivalent to the MHC-binding peptide described above, except that in this embodiment, the peptide is going to be used as a candidate antigenic peptide for binding to a T cell receptor) is randomly produced from a possible library of candidate antigenic peptides, so that each baculovirus in the population may express a different candidate antigenic peptide. In a preferred embodiment, each of the peptides in the library comprises: (1) conserved amino acid residues at specific positions in the sequence sufficient to enable the peptide to bind to the MHC molecule; and (2) randomly generated amino acid residues in the remaining positions in the sequence. As discussed above, this strategy reduces the number of peptide combinations required for the library to be sufficient to screen a T cell receptor. Various aspects of these recombinant baculoviruses and methods of production thereof have been discussed previously herein.

In one embodiment, the method further includes the step of infecting cells with the recombinant baculoviruses, so that an MHC-peptide molecule from the library of MHC-peptide molecules is displayed on the surface of each of the cells infected by the baculovirus. This method is useful for producing large libraries of functional MHC-peptide molecules displayed on the surface of baculovirus or baculovirus-infected cells that can be used in methods to identify antigenic peptides that bind to a specified T cell receptor. The antigenic peptide or peptides identified by such methods can then be used to identify the natural protein antigen that comprises such a peptide or in various other methods of monitoring the status of a T cell (i.e., in a disease state or vaccination protocol) or to design therapeutics for regulating the natural T cell receptor (e.g., to design agonists or antagonists of the identified peptide that can be used to regulate a T cell bearing that receptor in vivo or in vitro).

Another embodiment of the invention relates to a library of functional MHC-peptide molecules displayed on the surface of baculovirus or baculovirus-infected cells produced by the method of described above. Yet another embodiment of the invention relates to a population of cells infected with the recombinant baculoviruses produced by the method described above, wherein an MHC-peptide molecule from the library of MHC-peptide molecules is displayed on the surface of each of the cells infected by the baculovirus.

Accordingly, yet another embodiment of the present invention relates to a method for identifying baculovirus or baculovirus-infected cells that display an MHC-peptide complex that is recognized by a specific T cell receptor. More specifically, the method includes a first step of: (a) providing baculoviruses or baculovirus-infected cells that display on the baculoviral surface or cell surface, respectively, at least one MHC-peptide complex, wherein the complex comprises:

(1) at least a portion of an MHC molecule sufficient to form a peptide binding groove; and

(2) a candidate antigenic peptide that is covalently linked to the MHC molecule by a peptide linker and which is bound to the peptide binding groove of the MHC molecule, wherein the candidate antigenic peptide is from a library of candidate antigenic peptides, wherein each of the peptides in the library comprises conserved amino acids in a specific sequence sufficient to enable the peptide to bind to the MHC molecule.

In one embodiment, the library of candidate antigenic peptides represents from about 10³ to about 10⁹ different candidate antigenic peptides.

The method includes additional steps of: (b) contacting the baculoviruses or baculovirus-infected cells with a target T cell receptor; and (c) selecting baculoviruses or baculovirus-infected cells that bind to the target T cell receptor.

In general, in order to isolate the best candidate peptides for binding to a T cell receptor, it is desirable to repeat the selection process in additional cycles. Therefore, in one embodiment, the method can additionally include the steps of: (d) isolating the selected baculoviruses or baculoviruses from the selected baculovirus-infected cells of step (c); (e) infecting previously uninfected host cells with the isolated baculoviruses of (d) to produce baculoviruses or baculovirus-infected cells enriched for MHC-peptide complexes that bind to the target T cell receptor; (f) contacting the baculoviruses or baculovirus-infected cells from (e) with the target T cell receptor; and (g) selecting baculoviruses or baculovirus-infected cells that bind to the target T cell receptor. This method can be additionally extended by isolating the selected baculoviruses or the baculoviruses from the selected baculovirus-infected cells of step (g) and repeating steps (e)-(g) at least one additional time or as needed to isolate and identify an MHC-peptide complex that binds to the target T cell receptor.

In this method of the invention, the target T cell receptor is a T cell receptor for which it is desired to identify the peptide epitope recognized by the receptor. In one aspect, the target T cell receptor is from a patient with a T cell-mediated disease, such as an autoimmune disease or a hyperproliferative disease. In other embodiments, the target T cell receptor is from a patient with a different condition, such as an infection by a pathogenic microorganism or a patient with cancer. Knowledge of the antigen that is bound by a specified T cell can have therapeutic value for a variety of reasons. Preferably, the T cell receptor is an αβ T cell receptor. An αβ T cell (expressing an αβ T cell receptor) is a lineage of T lymphocytes found in mammalian species and birds that expresses an antigen receptor (i.e., a TCR) that includes an α chain and a β chain. According to the present invention, the terms “T lymphocyte” and “T cell” can be used interchangeably.

The T cell receptor can be expressed by a cell or provided as a soluble T cell receptor. In the former embodiment, the T cell receptor can be expressed by the T cell that naturally expresses the receptor (e.g., a T cell clone or hybridoma) or by another cell that recombinantly expresses the T cell receptor. In the latter embodiment, the soluble T cell receptor is preferably immobilized on a substrate or solid support for contact with the MHC-peptide library.

Briefly, a substrate or solid support refers to any solid organic supports, artificial membranes, biopolymer supports, or inorganic supports that can form a bond with a soluble T cell receptor without significantly affecting the ability of the T cell receptor to bind to an MHC-peptide complex for which the T cell receptor has specificity. Exemplary organic solid supports include polymers such as polystyrene, nylon, phenol-formaldehyde resins, acrylic copolymers (e.g., polyacrylamide). Exemplary biopolymer supports include cellulose, polydextrans (e.g., Sephadex®), agarose, collagen and chitin. Exemplary inorganic supports include glass beads (porous and nonporous), stainless steel, metal oxides (e.g., porous ceramics such as ZrO₂, TiO₂, Al₂O₃, and NiO) and sand. Soluble T cell receptors can be bound to a solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Cross-linking to a solid support involves forming a chemical bond between a solid support and the T cell receptor. Cross-linking commonly uses a bifunctional or multifunctional reagent to activate and attach a carboxyl group, amino group, sulfur group, hydroxy group or other functional group of the receptor to the solid support. Entrapment of involves formation of, inter alia, gels (using organic or biological polymers), vesicles (including microencapsulation), semipermeable membranes or other matrices, such as by using collagen, gelatin, agar, cellulose triacetate, alginate, polyacrylamide, polystyrene, polyurethane, epoxy resins, carrageenan, and egg albumin.

The target T cell receptor can be labeled with a detectable label. Detectable labels suitable for use include any compound detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵i, ³⁵s, ¹⁴C, or ³²p), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

As used herein, “TcR recognition” refers to the ability of a TcR to bind to an MHC-peptide complex, wherein the level of binding, as measured by any standard assay (e.g., an immunoassay or other binding assay), is statistically significantly higher than the background control for the assay. Binding assays are well known in the art. For example, a BlAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Other suitable assays for measuring the binding of one protein to another include, for example, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichrosim, or nuclear magnetic resonance (NMR).

In one embodiment, one can additionally measure whether a T cell receptor that is expressed by a T cell, when bound by an MHC-peptide complex produced by the invention, displays a T cell response to the binding. A T cell response occurs when a TCR recognizes an MHC protein bound to an antigenic peptide, thereby altering the activity of the T cell bearing the TCR. As used herein, a “T cell response” can refer to the activation, induction of anergy, or death of a T cell that occurs when the TCR of the T cell is bound by an MHC-peptide complex. As used herein, “activation” of a T cell refers to induction of signal transduction pathways in the T cell resulting in production of cellular products (e.g., interleukin-2) by that T cell. “Anergy” refers to the diminished reactivity by a T cell to an antigen. Activation and anergy can be measured by, for example, measuring the amount of IL-2 produced by a T cell after and MHC-peptide complex has bound to the TcR. Anergic cells will have decreased IL-2 production when compared with stimulated T cells. Another method for measuring the diminished activity of anergic T cells includes measuring intracellular and/or extracellular calcium mobilization by a T cell upon engagement of its TCR's. As used herein, “T cell death” refers to the permanent cessation of substantially all functions of the T cell. In the method of the present invention, the T cell will typically encounter the MHC-peptide complex in the absence of additional costimulatory signals that are normally required to induce T cell activation events. However, under some conditions, some type or level of T cell response will be measurable.

The ability of a T lymphocyte to respond to binding by an MHC-peptide complex can be measured by any suitable method of measuring T cell activation. Such methods are well known to those of skill in the art. For example, after a T cell has been stimulated with an antigenic or mitogenic stimulus, characteristics of T cell activation can be determined by a method including, but not limited to: measuring the amount of IL-2 produced by a T cell (e.g., by immunoassay or biological assay); measuring the amount of other cytokines produced by the T cell (e.g., by immunoassay or biological assay); measuring intracellular and/or extracellular calcium mobilization (e.g., by calcium mobilization assays); measuring T cell proliferation (e.g., by proliferation assays such as radioisotope incorporation); measuring upregulation of cytokine receptors on the T cell surface, including IL-2R (e.g., by flow cytometry, immunofluorescence assays, immunoblots); measuring upregulation of other receptors associated with T cell activation on the T cell surface (e.g., by flow cytometry, immunofluorescence assays, immunoblots); measuring reorganization of the cytoskeleton (e.g., by immunofluorescence assays, immunoprecipitation, immunoblots); measuring upregulation of expression and activity of signal transduction proteins associated with T cell activation (e.g., by kinase assays, phosphorylation assays, immunoblots, RNA assays); and, measuring specific effector functions of the T cell (e.g., by proliferation assays, cytotoxicity assays, B cell assays). Methods for performing each of these measurements are well known to those of ordinary skill in the art, and all such methods are encompassed by the present invention.

The present invention also includes any therapeutic, diagnostic, or research methods using peptides identified by the methods and tools described herein.

The following examples are provided for the purpose of illustration and are not intended to limit the scope of the present invention.

### EXAMPLES

### Example 1

The following example demonstrates the production and use of a peptide library to identify MHC Class 11-presented epitopes for specific T cell receptors.

To test the methodology of the present invention, the present inventors used two T cell hybridomas, both prepared from IA^{b} mice immunized with the peptide, p3K. This peptide binds well to IA^{b} (Rees et al., 1999, Proc. Natl. Acad Sci. USA 96:9781-9786) and its crystal structure bound to IA^{b} has been determined (Liu et al., 2002, Proc. Natl. Acad. Sci. USA 99:8820-8825).

The hybridoma, B3K-06 was produced from wild-type C57BL/6 mice immunized conventionally with the peptide (Rees et al., 1999, supra). Like most T cells resulting from immunization with a foreign peptide, it responds to IA^{b}-expressing APCs in the presence, but not in the absence of the peptide p3K (data not shown). It does not respond to APC expressing other alleles of the IA MHC Class II molecule. Also, as is commonly seen with conventional T cells, the interaction of the αβ TcR of B3K-06 with IA^{b}-p3K is very sensitive to changes in any of the peptide amino acids exposed on the surface of the IA^{b}-p3K complex. Mutation of positions Q2, K3, K5, N7 or K8 to alanine virtually eliminates recognition of p3K by B3K-06 (Liu et al., 2002, supra and additional data not shown).

The mouse T cell hybridoma, referred to as YAe-62, was chosen as a representative of broadly reactive T cells present in mice carrying transgenes and gene knockouts that lead to expression of MHC Class II that are almost completely occupied by a single peptide (Ignatowicz et al., 1996, Cell 84:521-529). The T cell was produced by immunization with p3K bound to IA^{b} in mice that express IA^{b} covalently linked to Eα, a dominant IA^{b} binding peptide derived from the MHC Class II IEα chain. YAe-62 responds to IA^{b}-p3K, but not to APCs lacking MHC Class II or to the IA^{b}-pEα APCs from the mouse from which it was derived. YAe-62 is also reactive against many cell types bearing IA^{b} in the absence of p3K. It also responds to APCs from a variety of mice carrying other alleles of IA. The inventors have postulated that this T cell responds mainly to the evolutionarily conserved regions of the IA molecule with less dependence on the peptide than seen with conventional T cells (Marrack et al., 2001, J. Immunol. 167:617-621). This property made this T cell a good candidate for an initial test of the method of the invention, since it could be predicted that many IA^{b}-peptide combinations should be found that bind to the selected T cell receptor. From the X-ray crystal structure of IA^{b} (Liu et al., 2002, supra) enough was known about how peptides bind to this MHC molecule to design a strategy in which the major MHC anchor amino acids of the peptide could be held constant and only five surface exposed amino acids need be varied in the library (FIG. 8).

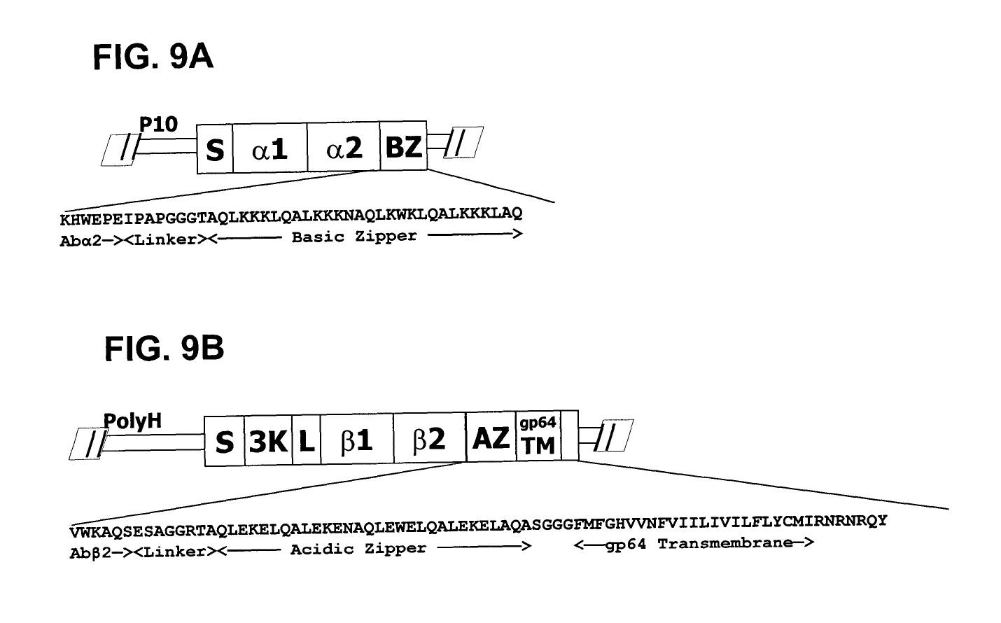

Methods were previously established that used baculovirus infected insect cells to produce soluble MHC molecules with covalently bound antigenic peptides (Crawford et al., 1998, Immunity, 8:675-682; Kozono et al., 1994, Nature, 369:151-154; Rees et al., 1999, Proc Natl Acad Sci USA, 96:9781-9786). These constructions were the starting point for developing insect cells displaying functional MHC Class II/peptides. Several modifications were made to constructs that encoded the mouse MHC Class II molecule, Ia^{b}, with various bound peptides. First, to increase the stability of the molecule, an acid/base leucine zipper (O'Shea et al., 1993, Current Biology, 3:658-667) was attached to the C-termini of the extracellular portions of the MHC α and β chains replacing what would normally be the transmembrane regions of these proteins. The basic half of the zipper was attached to the α chain (FIG. 9A) and the acidic half to the β chain (FIG. 9B). In addition, sequence encoding the transmembrane and cytoplasmic tail of the baculovirus major coat glycoprotein, gp64, was attached to the end of the acid zipper (FIG. 9B). SF9 insect cells infected with virus encoding this construction produced the MHCII molecule at a high level anchored on the cell surface (data not shown) via the gp64 transmembrane. Also, to make SF9 cells better APCs (Cai et al., 1996Proc Natl Acad Sci USA, 93:14736-14741), a version transfected with the genes for mouse ICAM and B7.1 was established (data not shown). When the ability of SF9 cells displaying the Ia^{b}-p3K complex to present the antigen to B3K-06 or Yae-62 was tested, the presence of ICAM/B7 greatly improved IL-2 production (data not shown). These results showed that Ia^{b}-p3K could be displayed on the surface of insect cells in a form easily recognized by T cells. Further, as described above, FIG. 3 shows the detection of displayed IA^{b}-p3K on infected SF9 insect cells, and the functionality of the displayed MHC/peptide complex was shown by the stimulation of T cell hybridomas with receptors of known MHC/peptide specificity (FIG. 4).

Next fluorescent, soluble αβTCR reagents were prepared for use in flow cytometry to detect insect cells displaying the appropriate MHCII/peptide combination. Fluorescent multivalent versions of the soluble αβTCR's of B3K-06 and YAe-62 bound to insect cells displaying the IA^{b}-p3K, but not a control MHCII-peptide combination (data not shown).

Insect cells displaying IA^{b}-p3K bound the αβTCR reagents very heterogeneously, probably due to heterogeneous expression of IA^{b}-p3K due to variations in the multiplicity of infection and the lack of synchrony in viral infection and expression. To focus on cells bearing a particular level of IA^{b}, the cells were stained simultaneously with the fluorescent αβTCR reagents and with an anti-IA^{b} Mab that did not interfere with αβTCR binding. In this case, there was a direct correlation between the amount of surface IA^{b}-p3K expressed by an individual insect cell and the amount of αβTCR bound with cells bearing a particular level of IA^{b}-p3K binding the αβTCRs uniformly (data not shown). Therefore, comparing the two types of staining gave a useful tool to evaluate the relation between peptide sequence and the strength of αβTCR binding (see below).

The experiments showed that fluorescent αβTCRs could be used with flow cytometry to identify insect cells infected with a baculovirus encoding a specific MHC/peptide combination. The inventors next tested whether this system could be used to enrich baculoviruses encoding a particular MHC/peptide. Insect cells were infected at a multiplicity of infection (MOI) of about one with a mixture of baculoviruses. One percent of the viruses encoded the IA^{b}-p3K molecule and 99% encoded a control molecule (an αβTCR β chain). The infected cells were stained with fluorescent YAe-62-αβTCR and analyzed by flow cytometry. Although a distinct population of brightly fluorescent cells was not seen, the 1% of the cells with the brightest fluorescence were sorted as were an equal number of cells which were very dully fluorescent (data not shown). The recovered infected cells were cultured with fresh insect cells to produce new viral stocks. These stocks were used to infect insect cells that were tested again with the fluorescent αβ TCR reagent. The cells infected with virus from the few fluorescent positive cells in the original population were now nearly all brightly fluorescent and those infected with the virus from the fluorescently dull cells were nearly all negative for binding of the αβTCR (data not shown). These results showed that flow cytometry could be used with a fluorescent multimerized a PTCR to find and greatly enrich insect cells infected with a virus encoding a specific MHC/peptide combination.

The most widely used method for introducing gene constructions into baculovirus involves assembling the construct first in an E. coli transfer plasmid where it is flanked by sections of baculovirus DNA. The complete construct is then introduced into baculovirus by homologous recombination using any of the commercially available modified baculovirus DNAs that require homologous recombination with the plasmid in order to generate functional circular viral DNA (Kitts and Possee, 1993, Biotechniques, 14:810-817). Based on this procedure, an IA^{b} peptide library was constructed in two steps. In the original transfer plasmid that encoded the displayed IA^{b}-p3K, the site encoding the peptide was flanked with unique restriction sites, one in the section encoding the β chain leader and the other in the section encoding the linker from the peptide to the N-terminus of the β chain. The DNA between these sites was replaced with DNA encoding enhanced GFP in frame with the IA^{b} signal peptide and with a 3′ termination codon (FIG. 8A). Thus, cells infected with baculovirus carrying this construct produced GFP, but not an IA^{b} molecule, because of disruption of the IA^{b} β chain gene.

A peptide library was then designed based on the structure of p3K bound to IA^{b}. The inventors used oligonucleotides with random nucleotides in codons encoding five peptide amino acids (p2, p3, p5, p7 and p8) corresponding to the central surface exposed amino acids of p3K bound to IA^{b}. Other positions were kept identical to p3K, including alanines at the four standard anchor residues at p1, p4, p6, and p9. These oligonucleotides were used in a PCR to create a DNA fragment randomized in these five codons and with 5′ and 3′ end restriction enzyme sites compatible with those in the signal peptide and linker (FIG. 8B). This fragment was ligated into the restricted plasmid, replacing the GFP sequence and restoring a fuctional IA^{b} β chain gene (FIG. 8C). The mixture of plasmids was then used to transform E. coli and a bulk plasmid preparation was made. The plasmids were cotransfected with BaculoGold baculovirus DNA into SF9 insect cells to produce a mixed viral stock in which each virus carried the genes for IA^{b} with a different peptide bound. Although it is difficult to calculate the efficiency with which recombination yield infectious baculovirus, it was estimated that the size of this library was between 10⁴ and 10⁵ independent viruses.

A large number of SF9 insect cells were infected at an MOI of about one with baculovirus carrying the IA^{b} peptide library. After 3-4 days the cells were analyzed with fluorescent B3K-06 or YAe-62 soluble αβTCR, as described above. Fluorescent cells were sorted and cultured with fresh uninfected SF9 cells to create new infected cells for analysis and an enriched viral stock. This process was repeated 3 to 4 times. In each case, when no clear fluorescent population was apparent, the brightest 1% of the infected cells was sorted. In later rounds, the majority of the cells in a clearly distinguishable fluorescent population were sorted. Infected cells binding the B3K-06 αβTCR were apparent only after two rounds of enrichment but eventually yielded a population with uniform binding (data not shown). Infected cells that bound the YAe-62 αβTCR were detectable even with the initial library of viruses and enriched rapidly to yield a population with more heterogeneous levels of binding to the receptor (data not shown).

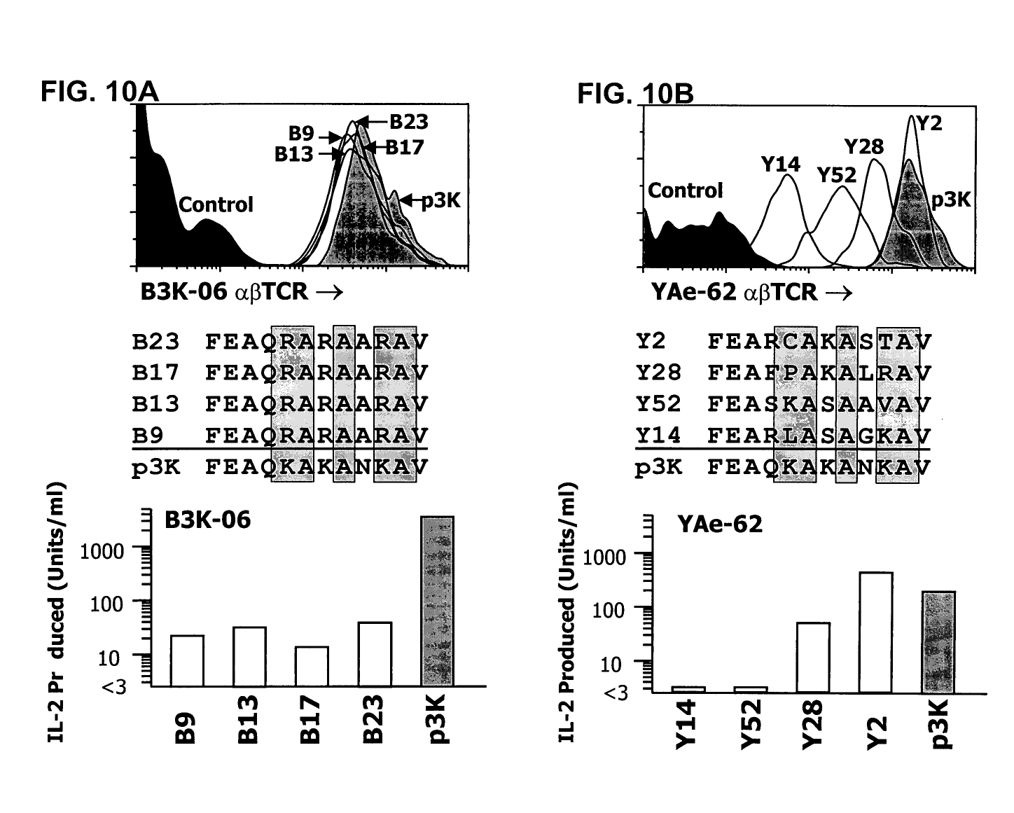

At the time of the final enrichment, single infected cells binding each of αβTCRs were sorted into individual wells of 96 well culture plates containing fresh SF9 cells in order to prepare clonal viral stocks. These stocks were used to infect fresh SF9 cells which were reanalyzed for binding to the appropriate αβTCR as described above. Viral DNA from the clones that showed homogeneous TCR binding at a particular level of IA^{b} were used as template in a PCR using oligonucleotides that flanked the peptide site in the construct and a third internal oligonucleotide was used to sequence the PCR fragment. The majority of PCR fragments yielded a single unambiguous peptide sequence. These viruses were used to infect SF9 cells that expressed mouse ICAM and B7.1. The infected cells were used as APCs for either the B3K-06 or YAe-62 hybridoma with IL-2 production being a measure of IA^{b}-peptide recognition. Viruses expressing IA^{b}-peptide combinations that neither bound to the αβTCR nor stimulated the T cell hybridomas were used as negative controls and virus producing IA^{b}-p3K was used as the positive control. Results with a few representative virus clones are shown in FIG. 10A and 10B, and a summary of all of the results are shown in Table 1.

TABLE 1

| | | B3K-06 TCR | IL-2 |
|---|---|---|---|
| No. of | Peptide Sequence | Binding | Production |
| Clones | 1 2 3 4 5 6 7 8 9 | (% of p3K) | (units/ml) |

| | | | | |
|---|---|---|---|---|
| 42 | F E A Q R A R A A R A V | 66.8 | 25 | SEQ ID NO:16 |
| p3K | F E A Q K A K A N K A V | 100.0 | 3500 | SEQ ID NO:17 |
| pEα | F E A Q G A L A N I A V | 0.4 | <3 | SEQ ID NO:18 |

| | | | |
|---|---|---|---|
| | | YAe-62 TCR | IL-2 |
| No. of | Peptide Sequence | Binding | Production |
| Clones | 1 2 3 4 5 6 7 8 9 | (% of p3K) | (units/ml) |

| | | | | |
|---|---|---|---|---|
| 5 | F E A L Y A K A L T A V | 98.7 | 1717 | SEQ ID NO:19 |
| 4 | F E A R C A K A S T A V | 102.5 | 467 | SEQ ID NO:20 |
| 3 | F E A F M A R A K A A V | 107.5 | 1256 | SEQ ID NO:21 |
| 3 | F E A L P A R A A A A V | 70.4 | 681 | SEQ ID NO:22 |
| 2 | F E A H T A L A P R A V | 80.4 | 6 | SEQ ID NO:23 |
| 1 | F E A S L A R A R S A V | 76.2 | 5 | SEQ ID NO:24 |
| 1 | F E A Y T A R A R T A V | 58.3 | 5 | SEQ ID NO:25 |
| 1 | F E A Y T A R A R T A V | 54.9 | 7 | SEQ ID NO:26 |
| 1 | F E A T T A R A L T A V | 52.0 | 6 | SEQ ID NO:27 |
| 1 | F E A E K A K A L T A V | 49.6 | 9 | SEQ ID NO:28 |
| 1 | F E A Q V A H A L P A V | 48.6 | 32 | SEQ ID NO:29 |
| 1 | F E A F P A K A L R A V | 38.5 | 47 | SEQ ID NO:30 |
| 1 | F E A L S A K A N T A V | 33.3 | <3 | SEQ ID NO:31 |
| 1 | F E A R E A K A L A A V | 27.0 | <3 | SEQ ID NO:32 |
| 1 | F E A A L A R A V P A V | 23.4 | <3 | SEQ ID NO:33 |
| 1 | F E A S K A S A A V A V | 13.0 | <3 | SEQ ID NO:34 |
| 1 | F E A R L A S A G K A V | 2.6 | <3 | SEQ ID NO:35 |
| 1 | F E A E R A R A A S A V | 2.3 | <3 | SEQ ID NO:36 |
| 1 | F E A R T A H A R N A V | 1.4 | <3 | SEQ ID NO:37 |
| 1 | F E A P Y A Q A P H A V | 1.3 | <3 | SEQ ID NO:38 |
| p3K | F E A Q K A K A N K A V | 100.0 | 205 | SEQ ID NO:17 |
| pEα | F E A Q G A L A N I A V | 0.3 | <3 | SEQ ID NO:18 |

Given the previous data indicating that the B3K-06 αβTCR interacted with all five of the p3K amino acids varied in this library (Liu et al., 2002, Proc Natl. Acad Sci USA, 99:8820-8825) and data not shown, it was expected that mimotopes satisfying this receptor would be infrequent or perhaps even absent in the library. Indeed, only one peptide was recovered from the library with the B3K-06 αβTCR, FEAQRARAARVD (SEQ ID NO: 10). It was found in all 42 clones analyzed with unambiguous αβTCR binding and peptide sequence. The sequence of this peptide was strikingly similar to that of p3K. Like p3K, it had a glutamine at position 2. It had arginines at positions 3, 5 and 8 corresponding to the lysines found in these positions in p3K, most likely reflecting the importance of the positive charges at these positions. Since there are six codons for arginine and only two for lysine, it is not surprising that in the relatively small library used in these experiments, arginines would be more likely to be found than lysines. The most significant between this peptide and p3K was an alanine instead of asparagine found at position 7. When bound to IA^{b} on B7.7/ICAM expressing SF9 APCs, FEAQRARAARVD (SEQ ID NO:10) was able to stimulate B3K-06 to produce IL-2, but not nearly as well as did p3K. This loss of stimulating activity was caused by one or more of the lysine to arginine substitutions and/or the asparagine to alanine substitution at p7. Interestingly, the substitution of alanine for asparagine in p3K, eliminated the response of B3K-06 to soluble peptide presented by an IA^{b} bearing mouse APC (data not shown). Perhaps the very high density of IA^{b}-peptide on the surface of the insect cells allows for responses to peptides that would normally not be stimulatory with peptides presented by conventional APCs. Strikingly, despite the very great difference in their abilities to stimulate IL-2 production, IA^{b} complexed with the library-derived peptide bound the B3K-06 αβTCR only slightly less well than did the IA^{b}-p3K complex. This observation was made as well with IA^{b}-peptide combinations enriched with the YAe-62 αβTCR and is discussed in more detail below.

Consistent with the prediction that the αβTCR of YAe-62 would be more peptide promiscuous than that of B3K-06, 20 different peptide sequences were found among the analyzed clones that produced an IA^{b}d-peptide combination that bound the YAe-62 αβTCR. It is likely that many more would be identified if more clones were analyzed. Five sequences were found multiple times. Not unexpectedly, these were among those that bound the YAe-62 αβTCR most strongly. There was a one hundred fold range in the intensity of αβTCR binding to the different IA^{b}-peptide combinations ranging from about 4 fold to 400 fold binding above that seen with a negative control peptide. One obvious property of these peptides stands out. There was a very strong selection for an amino acid at position 5 with a potential positive change. In 16 of 20 of the peptides a lysine, arginine or histidine was found at position 5 matching the lysine found in p3K. The other four had one of these amino acids at position 3 or 8 matching either of the lysines at these other positions in p3K.

Overall, however, there was no strong selection for amino acids homologous to those of p3K at positions 2, 3, 7 or 8. The amino acids at positions 2 and 3 appear nearly random, suggesting little or not essential contact between this part of the MHC-peptide ligand and the receptor, although these positions may contribute to the wide range of apparent αβTCR affinities seen. While not homologous to the asparagine in p3K, there was an over-representation of leucine at position 7 in the selected peptides. The amino acid in this position is only partially exposed on the surface and can contribute significantly to peptide-MHC interaction (Liu et al., 2002, Proc Natl. Acad Sci USA, 99:8820-8825). After asparagine, leucine is the most common amino acid found at this position in peptides found naturally bound to IA^{b} (Dongre et al., 2001, Eur J Immunol, 31:1485-1494; (Liu et al., 2002, Proc Natl. Acad Sci USA, 99:8820-8825). On the other hand, the amino acid at position 8 is predicted to be fully surface exposed. In the selected peptides, rather than an amino acid homologous to the lysine of p3K, there is a clear over representation of amino acids with small neutral side chains (threonine, serine, alanine, glycine) at this position. Perhaps this indicates that in general larger side chains can be inhibitory at this position.

The 12 IA^{b}-peptide combinations that bound the YAe-62 αβTCR most strongly were also the ones that were able to induce IL-2 production from YAe-62. However, as was the case with the B3K-06 selected peptide, among these stimulating peptides there was no direct correlation between the amount of IL-2 produced and the strength of binding to αβTCR. For example, IA^{b} bearing either FEAQTAKARGAVD (SEQ ID NO:39) or FEALPARAAAAVD (SEQ ID NO:40) bound the YAe-62 αβTCR nearly equally well, but there was a 100 fold difference in the amount of IL-2 production that they stimulated. Possible explanations for this dichotomy between apparent affinity and IL-2 production are discussed below.

Overall, the results supported the original prediction that for conventional T cells, such as B3K-06, most of the surface exposed residues of the peptide would be important in MHC-peptide recognition, while for broadly, allo-MHC reactive T cells such as YAe-62, peptide recognition would be much more promiscuous.

### Example 2

The following example demonstrates the production and use of a peptide library to identify MHC Class I-presented epitopes for a specific T cell.

The inventors have previously shown that one can covalently attach peptides to MHC Class I via a flexible linker to the N-terminus of the β2m chain of the molecule (White et al., 1999, J Immunol 162:2671-2676). This method has been adapted using the methods of the present invention to display MHC Class I on baculovirus and baculovirus insect cells. The previously described construct to produce soluble MHC Class I (White et al., ibid.) was modified to add the baculovirus GP64 transmembrane to the heavy chain of the molecule just after the alpha3 domain (FIG. 11A). The initial attempt was made with the D^{d} MHC Class I molecule of mouse. As previously described (White et al., ibid.), a dominant D^{d} binding HIV gp120 peptide (PHIV) was attached to the N-terminus of β2m via a flexible linker (FIG. 11B). SF9 insect cells infected with baculovirus carrying this construct according to the method of the invention express the D^{d}-pHIV on their surface (FIG. 12A) and this complex can be recognized by a T cell specific for this combination (FIG. 12B).

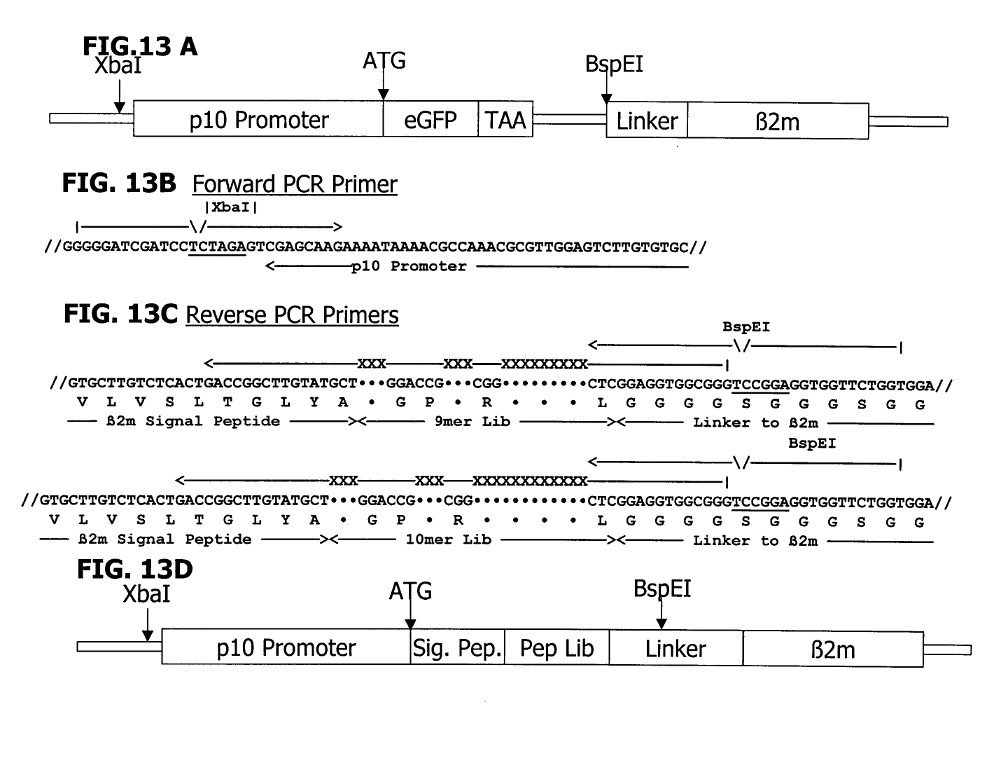

The strategy to produce a library of D^{d}-peptides was similar to that used for constructing MHC Class II peptide libraries described in Example 1 (FIG. 13A). The β2m gene was disrupted by sequence encoding enhanced GFP (FIG. 13A). Since the peptide binding motif of D^{d} is well-understood, oligonucleotides were used that fixed the four peptide anchor amino acids (glycine, proline, arginine and leucine). Codons for other positions were randomized. Forward (FIG. 13B) and reverse (FIG. 13C) oligonucleotide primers were used to construct a PCR fragment that encoded peptides that could bind to D^{d}. Two different reverse primer oligonucleotides were used that allowed the total length of the peptide to be either 9 or 10 amino acids (FIG. 13C). Referring to FIG. 13C, positions 2,3,5 and the C-terminal amino acid of the peptide was held constant as glycine, proline, arginine and leucine, while other positions were randomized. The oligonucleotides were used to synthesize a DNA fragment that had restriction enzyme sites that allowed cloning in front of the β2m gene, replacing a GFP stuffer. The restricted fragment was ligated into an E. coli plasmid containing the genes for D^{d} heavy chain and β2m (FIG. 13D). The mixture of ligated plasmids was incorporated into baculovirus by standard recombination techniques. The estimated the size of library produced was about 10⁴ to 10⁵.

To screen the library, soluble a αβTCR were produced from a mouse T cell specific for D^{d} plus an unknown self-peptide (Endres et al., 1983, J Immunol 131:1656-1662). A multimeric, fluorescent version of the αβTCR was produced as described for MHCII specific αβTCRs. SF9 cells, infected with the library at a multiplicity of infection (MOI) of <1, were analyzed for binding of the fluorescent αβTCR (data not shown). Although no clearly fluorescent population of cells was seen, of those with good surface D^{d} expression, the 1% of the cells with the brightest fluorescence were sorted and cultured with fresh insect cells to expand the virus. This type of enrichment was repeated six times, producing a clear population of infected cells was detected that bound the αβTCR (data not shown). The infected cells were cloned with fresh insect cells to prepare clonal viral stocks. These stocks were re-tested for encoding a D^{d}-peptide combination that bound the αβTCR.

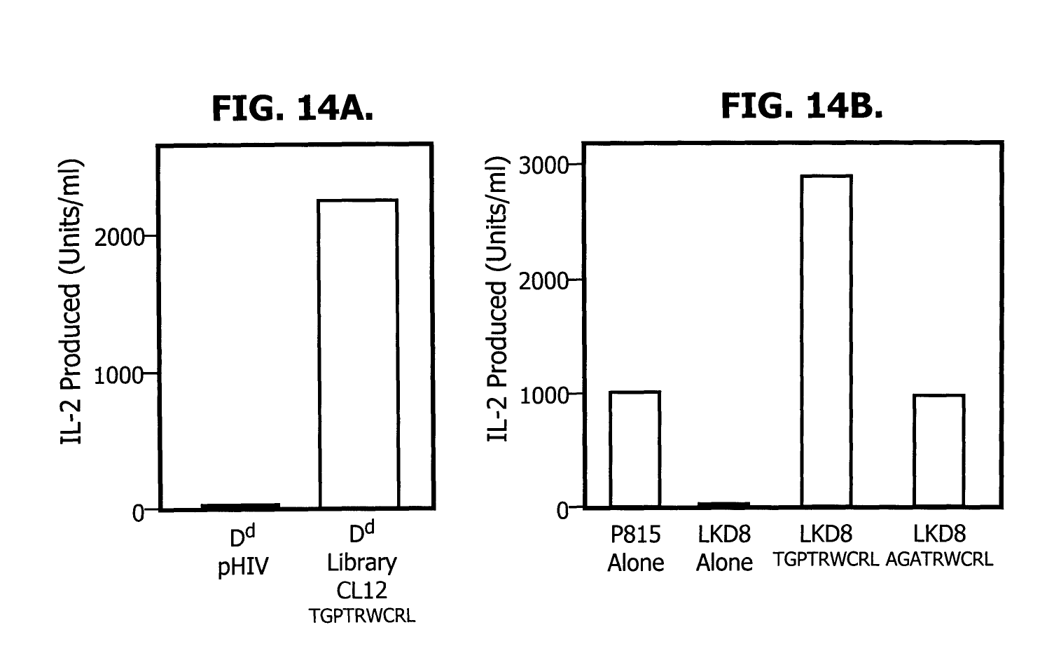

DNA from a number of these clones was sequenced through the region encoding the peptide to determine the peptide sequence. Only one sequence was found, a 9mer, TGPTRWCRL (represented by SEQ ID NO:50; the underlined amino acids are in the positions varied in the library). Infected insect cells expressing D^{d} bearing this peptide, when tested as antigen presenting cells, specifically stimulated IL-2 production from the original T cell donor of the αβTCR (FIG. 14A). A search of the mouse genome for proteins that contained peptides similar to the library peptide yielded a very similar sequence (AGATRWCRL; SEQ ID NO:51) intheprotein, spin(GenBank Accession No. BC011467). The library peptide and the spin peptide were synthesized and tested with a D^{d} expressing, Tap deficient, cell line for recognition by the original T cell (FIG. 14B). Referring to FIG. 14B, two mouse cell lines were used as antigen presenting cells: 1) P815, a DBA/2 derived mastocytoma, that was one of the cell lines originally used to demonstrate that the target of 3DT-52.5 was D^{d} plus a bound unknown self-peptide and 2) LKD8, a mouse cell line that expresses D^{d}, but cannot load peptides due to a defect in antigen processing. In the case of LKD8 the cell line was tested alone or in the presence of 100 ug/ml of the library derived peptide, TGPTRWCRL (SEQ ID NO:50), or a peptide derived from the spin protein, AGATRWCRL (SEQ ID NO:51). After twenty four hours the culture supernatants were assayed for IL-2. Without an added peptide, the D^{d} on this cell line was not recognized because the Tap deficiency prevents loading of endogenous peptides. Synthetic versions of both the library peptide and the spin peptide, but not the D^{d} binding peptide from HIV, restored the ability of the cells to stimulate the T cells, suggesting that spin may the source of the unknown peptide recognized by this T cell.

This approach should be generalizable to other MHC Class I molecules and will be useful in identification of unknown or modified MHC Class I epitopes in cancer immunotherapy.

### Example 3

The following example describes the production of larger libraries by direct cloning into baculovirus DNA.

In prior experiments, the inventors have worked with small libraries (10⁴-10⁵) prepared by introducing the library of into baculovirus via an E. coli transfer plasmid intermediate. The inventors have now developed methods that allow the cloning of the randomized PCR DNA fragment directly into baculovirus DNA already carrying the MHC Class I or MHC Class II genes. The principle is to clone via homing endonucleases that recognize extremely rare DNA sequences and cut the DNA leaving non-palindromic 4 base 3′ protruding ends. Compatible ends can be generated on the PCR fragment using a conventional restriction enzyme, such as BstXI. Although other rare cutting conventional restriction enzymes can be used, using enzymes that leave non-palindromic ends has the advantage that during ligation competing reactions (fragment to fragment or vector to vector) are eliminated.

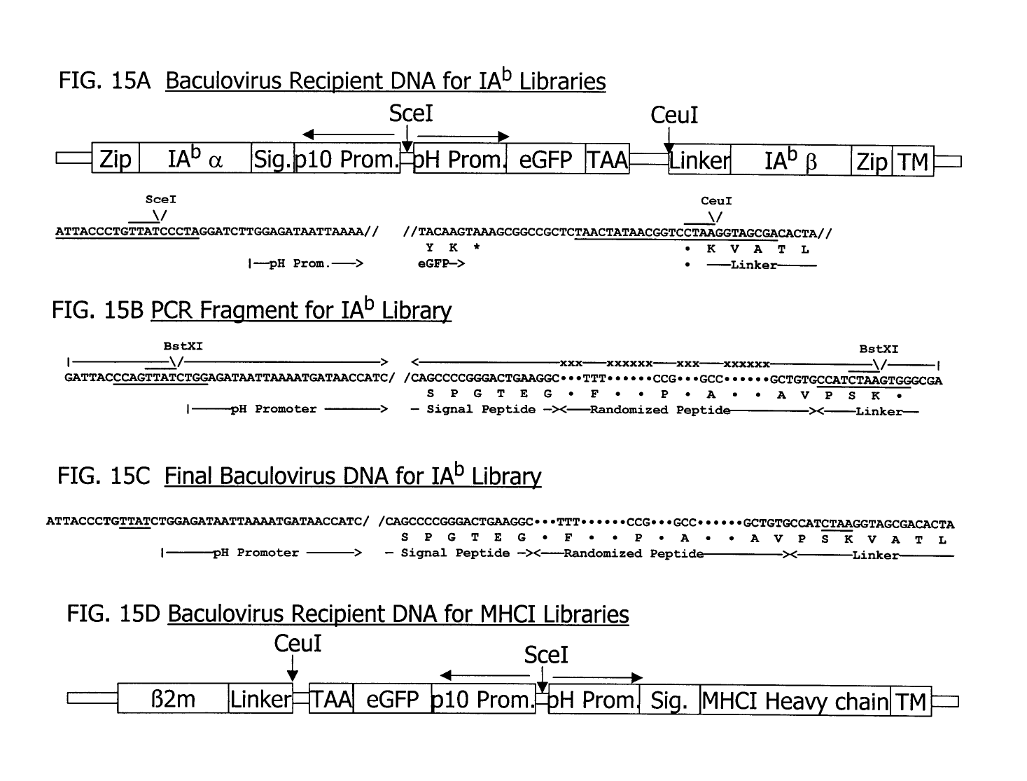

To test this idea, the inventors constructed the mouse IA^{b} peptide library, altering the E. coli transfer plasmid construct for display of IA^{b} with a covalently attached peptide (FIG. 15A). A site for the enzyme, Ceul, was placed in the region encoding the linker between the peptide and β chain. A site for the enzyme SceI was introduced just upstream of the polyhedrin promoter. Sequence encoding the peptide was replaced with sequence encoding eGFP. The construct was introduced into baculovirus by the standard recombination method. Infection of insect cells with the resulting virus resulted in expression of easily detectable GFP, but no surface IA^{b}, because of the disruption of the β chain gene by that of eGFP. Baculovirus DNA containing the construct was purified and digested with CeuI and Scel to release the portion encoding the GFP gene. A DNA fragment was prepared by PCR that encoded the baculovirus polyhedrin promoter, the Ab beta chain signal peptide, and an Ab binding peptide randomized at 6 positions exposed on the surface of the IA^{b}/peptide complex. BstXI sites were introduced at the ends of the fragment such that restricting the fragment with BstXI generated protruding ends compatible with SceI and CeuI (FIG. 15B). When this fragment was ligated into the CeuI/SceI digested baculovirus DNA, the IA^{b} beta gene was restored with linked sequence encoding the library of peptides (FIG. 15C). The competing reaction in this ligation is the reintroduction of the released GFP gene fragment. This reaction is held to a minimum by using a 4-8 fold molar excess of the PCR fragment during the ligation. Furthermore, reintroduction of the GFP yields a virus that produces green infected cells, which can easily be avoided during screening of the library.

Transfection of the ligated DNA into SF9 insect cells led to the appearance of IA^{b} expressing insect cells at a frequency of ˜10% (data not shown). Therefore, without any further modification, libraries of 10⁷ members can be generated by transfection of 10⁸ SF9 cells. The inventors have now adapted the MHC Class I β2m construct described in Example 2 to incorporate sites for these homing enzymes, so that a similar strategy can be used for MHC Class I peptide libraries (FIG. 15D).

Any of the references disclosed below or elsewhere herein are incorporated herein by reference in their entireties.

References:

Boublik et al. (1995) Biotechnology (NY) 13, 1079-1084

Ernst et al. (1998) Nucleic Acids Res 26, 1718-1723

Grabherr and Ernst (2001) Comb Chem High Throughput Screen 4, 185-192

Grabherr et al. (2001) Trends Biotechnol 19, 231-236

Kappler et al. (1994) Proc Natl Acad Sci USA 91, 8462-8466

Kozono et al. (1994) Nature 369, 151-154

Liu et al. (2002) Proc Natl Acad Sci USA 99, 8820-8825

O'Shea et al. (1993) Current Biology 3, 658-667

White et al. (1999) J Immunol 162, 2671-2676

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A recombinant baculovirus expression vector for expression of functional MHC-peptide molecules, comprising a baculovirus genome comprising:
a) a first nucleic acid sequence inserted into a first baculovirus structural gene at a position under control of a promoter for the first baculovirus structural gene, wherein the first nucleic acid sequence encodes at least a portion of the extracellular domains of the α chain of a major histocompatibility complex (MHC) Class I molecule or at least a portion of the extracellular domains of the α chain of a MHC Class II molecule;
b) a second nucleic acid sequence inserted into a second baculovirus structural gene at a position under control of a promoter for the second baculovirus structural gene, wherein the second nucleic acid sequence encodes at least a portion of the extracellular domains of:
i) a β2-microglobulin (β2m) chain of a MHC Class I molecule if the first nucleic acid sequence encodes at least a portion of the extracellular domains of the α chain of a MHC Class I molecule; or
ii) a β chain of a MHC Class II molecule if the first nucleic acid sequence encodes at least a portion of the extracellular domains of the α chain of a MHC Class II molecule;
c) a third nucleic acid sequence encoding an MHC-binding peptide;
d) a fourth nucleic acid sequence encoding a peptide linker, wherein the third nucleic acid sequence encoding the MHC-binding peptide is connected to the 5′ end of the first or second nucleic acid sequence by the fourth nucleic acid sequence; and
e) a fifth nucleic acid sequence encoding at least a transmembrane region of a membrane protein, wherein the first or the second nucleic acid sequence is inserted into the baculovirus genome in frame with the fifth nucleic acid sequence, the fifth nucleic acid sequence being located after the 3′ end of the first or second nucleic acid sequence;
wherein the portion of the extracellular domains of the α chain of the MHC Class I molecule and the portion of the extracellular domains of the β2m chain of the MHC Class I molecule, or the portion of the extracellular domains of the α chain of the MHC Class II molecule and the portion of the extracellular domains of the β chain of the MHC Class II molecule, form a peptide binding groove of an MHC molecule, and wherein the MHC-binding peptide comprises a sequence of amino acids that binds to the peptide binding groove.

2. The recombinant baculovirus expression vector of claim 1, wherein the first nucleic acid sequence encodes at least a portion of the extracellular domains of the α chain of a MHC Class I molecule, and wherein the second nucleic acid sequence encodes at least a portion of the extracellular domains of a β2m chain of a MHC Class I molecule.

3. The recombinant baculovirus expression vector of claim 2, wherein the third nucleic acid sequence encoding the MHC-binding peptide is connected to the 5′ end of the second nucleic acid sequence encoding at least a portion of the extracellular domains of a β2m chain of a MHC Class I molecule by the fourth nucleic acid sequence encoding a peptide linker.

4. The recombinant baculovirus expression vector of claim 1, wherein the first nucleic acid sequence encodes at least a portion of the extracellular domains of the α chain of a MHC Class II molecule, and wherein the second nucleic acid sequence encodes at least a portion of the extracellular domains of a β chain of a MHC Class II molecule.

5. The recombinant baculovirus expression vector of claim 4, wherein the third nucleic acid sequence encoding the MHC-binding peptide is connected to the 5′ end of the second nucleic acid sequence encoding at least a portion of the extracellular domains of a β chain of a MHC Class II molecule by the fourth nucleic acid sequence encoding a peptide linker.

6. The recombinant baculovirus expression vector of claim 1, wherein the fifth nucleic acid sequence encodes at least the transmembrane portion of a membrane protein selected from the group consisting of: baculovirus envelope protein gp64, MHC Class I, MHC Class II, and p26.

7. The recombinant baculovirus expression vector of claim 1, wherein the fifth nucleic acid sequence encodes at least the transmembrane portion of baculovirus envelope protein gp64.

8. The recombinant baculovirus expression vector of claim 1, wherein the fifth nucleic acid sequence encodes a full-length gp64.

9. The recombinant baculovirus expression vector of claim 1, wherein the fifth nucleic acid sequence encodes only the transmembrane portion and cytoplasmic tail of gp64.

10. The recombinant baculovirus expression vector of claim 1, wherein the first nucleic acid sequence further comprises, 3′ of the nucleic acid sequence encoding the extracellular domains of the α chain of an MHC molecule, a nucleic acid sequence encoding a basic leucine zipper dimerization helix.

11. The recombinant baculovirus expression vector of claim 1, wherein the second nucleic acid sequence further comprises, 3′ of the nucleic acid sequence encoding the extracellular domains of the β chain of a Class II MHC molecule or the Class I β2m molecule, a nucleic acid sequence encoding an acidic leucine zipper dimerization helix.

12. The recombinant baculovirus expression vector of claim 1, wherein the peptide linker encoded by the fourth nucleic acid molecule comprises at least about 8 amino acid residues, wherein the linker facilitates the binding of the MHC-binding peptide to the peptide binding groove of the MHC molecule.

13. The recombinant baculovirus expression vector of claim 1, wherein the MHC-binding peptide is from a library of candidate antigenic peptides, wherein the each of the peptides in the library comprises conserved amino acids in a specific sequence sufficient to enable the peptide to bind to the peptide binding groove of the MHC molecule that is encoded by the vector.

14. The recombinant baculovirus expression vector of claim 1, wherein the MHC-binding peptide is from a library of candidate antigenic peptides, wherein each of the peptides in the library comprises between about 4 and 5 conserved amino acids in a specific sequence sufficient to enable the peptide to bind to the peptide binding groove of the MHC molecule that is encoded by the vector.

15. The recombinant baculovirus expression vector of claim 1, wherein the MHC-binding peptide is from a library of candidate antigenic peptides representing from between about 10³ and about 10⁹ different candidate antigenic peptides.

16. A recombinant baculovirus comprising the recombinant baculovirus expression vector of claim 1, wherein the recombinant baculovirus expresses and displays on its surface a functional MHC-peptide molecule encoded by the vector.

17. A population of cells infected with the recombinant baculovirus of claim 16, wherein the cells display the functional MHC-peptide molecules expressed by the baculovirus on their surfaces.

18. A recombinant insect cell that displays on its surface a functional MHC-peptide molecule, wherein the recombinant insect cell:
a) has been transfected with recombinant nucleic acid molecules that encode at least the extracellular domains of an MHC molecule, the recombinant nucleic acid molecules comprising:
i) a first nucleic acid sequence operatively linked to an expression control sequence, wherein the first nucleic acid sequence encodes at least a portion of the extracellular domains of the α chain of a major histocompatibility complex (MHC) Class I molecule or at least a portion of the extracellular domains of the α chain of a MHC Class II molecule; and
ii) a second nucleic acid sequence operatively linked to an expression control sequence under control of a baculovirus promoter and enhancer, wherein the second nucleic acid sequence encodes at least a portion of the extracellular domains of:
(1) a β2-microglobulin (β2m) chain of a MHC Class I molecule if the first nucleic acid sequence encodes at least a portion of the extracellular domains of the α chain of a MHC Class I molecule; or
(2) a β chain of a MHC Class II molecule if the first nucleic acid sequence encodes at least a portion of the extracellular domains of the α chain of a MHC Class II molecule;
wherein the portion of the extracellular domains of the α chain of the MHC Class I molecule and the portion of the extracellular domains of the β2m chain of the MHC Class I molecule, or the portion of the extracellular domains of the α chain of the MHC Class II molecule and the portion of the extracellular domains of the β chain of the MHC Class II molecule, form a peptide binding groove of an MHC molecule; and
b) has been infected with a recombinant baculovirus comprising a third nucleic acid sequence under control of a baculovirus promoter and comprising a signal sequence, wherein the third nucleic acid sequence encodes an MHC-binding peptide, wherein the MHC-binding peptide comprises a sequence of amino acids that binds to the peptide binding groove of the MHC Class I molecule or the MHC Class II molecule.

19. A method for production of libraries of functional MHC-peptide molecules displayed on the surface of baculovirus and baculovirus-infected cells, comprising:
a) producing a population of recombinant baculoviruses by introducing into the genome of the baculoviruses:
i) a first nucleic acid sequence encoding at least a portion of the extracellular domains of the α chain of a major histocompatibility complex (MHC) Class I molecule or at least a portion of the extracellular domains of the α chain of a MHC Class II molecule, wherein the first nucleic acid sequence is introduced into the baculovirus genome at a position under control of a promoter for a first baculovirus structural gene;
ii) a second nucleic acid sequence encoding at least a portion of the extracellular domains of:
(1) a β2-microglobulin (β2m) chain of a MHC Class I molecule if the first nucleic acid sequence encodes at least a portion of the extracellular domains of the α chain of a MHC Class I molecule; or
(2) a β chain of a MHC Class II molecule if the first nucleic acid sequence encodes at least a portion of the extracellular domains of the α chain of a MHC Class II molecule;
wherein the second nucleic acid sequence is introduced into the baculovirus genome at a position under control of a promoter for a second baculovirus structural gene; and
wherein the portion of the extracellular domains of the α chain of the MHC Class II molecule and the portion of the extracellular domains of the β chain of the Class II MHC molecule, or the portion of the extracellular domains of the α chain of the Class I MHC molecule and the portion of the extracellular domains of the β2m chain of the Class I MHC molecule, respectively, form a peptide binding groove;
iii) a third nucleic acid sequence encoding a candidate antigenic peptide, wherein the candidate antigenic peptide is randomly produced from a possible library of candidate antigenic peptides so that each baculovirus in the population may express a different candidate antigenic peptide, wherein each of the peptides in the library comprises:
(1) conserved amino acid residues at specific positions in the sequence sufficient to enable the peptide to bind to the MHC molecule; and
(2) randomly generated amino acid residues in the remaining positions in the sequence;
wherein the third nucleic acid sequence is introduced into the baculovirus genome before the 5′ end of the first or second nucleic acid sequence;
iv) a fourth nucleic acid sequence encoding a peptide linker, wherein the third nucleic acid sequence encoding a candidate antigenic peptide is connected to the first or second nucleic acid sequence by the fourth nucleic acid sequence;
v) a fifth nucleic acid sequence encoding at least the transmembrane portion of a membrane protein, the membrane protein-encoding sequence being in frame with and located after the 3′ end of the first or second nucleic acid sequence; and
b) expressing the nucleic acid sequences of (i)-(v) on the surface of each of the baculoviruses in the population, wherein expression of the nucleic acid sequences of (i)-(v) results in the production of at least a portion of an MHC molecule which is covalently linked to the candidate antigenic peptide expressed by the given baculovirus via the peptide linker, and wherein the candidate antigenic peptide is bound to the peptide binding groove of the MHC molecule, thereby forming a library of MHC-peptide molecules displayed on the surface of baculoviruses, the library representing multiple different candidate antigenic peptides.

20. The method of claim 20, further comprising infecting cells with the recombinant baculoviruses, so that an MHC-peptide molecule from the library of MHC-peptide molecules is displayed on the surface of each of the cells infected by the baculovirus..

21. The method of claim 20, wherein the fifth nucleic acid sequence encodes at least the transmembrane portion of baculovirus envelope protein gp64.

22. The method of claim 20, wherein each of the peptides in the library comprises between about 4 and 5 conserved amino acids in a specific sequence sufficient to enable the peptide to bind to the MHC molecule.

23. The method of claim 20, wherein the nucleic acid sequences are introduced into the baculovirus genome using an E. coli transfer plasmid.

24. The method of claim 20, wherein the nucleic acid sequences are introduced into the baculovirus genome by direct cloning of the sequences into the genome.

25. The method of claim 20, wherein the library of candidate antigenic peptides represents from about 10³ to about 10⁹ different candidate antigenic peptides.

26. A library of functional MHC-peptide molecules displayed on the surface of baculovirus or baculovirus-infected cells produced by the method of claim 20.

27. A population of cells infected with the recombinant baculoviruses produced by the method of claim 20, wherein an MHC-peptide molecule from the library of MHC-peptide molecules is displayed on the surface of each of the cells infected by the baculovirus.

28. A method for identifying baculovirus or baculovirus-infected cells that display an MHC-peptide complex that is recognized by a specific T cell receptor, comprising:
a) providing baculoviruses or baculovirus-infected cells that display on the baculoviral surface or cell surface, respectively, at least one MHC-peptide complex, wherein the complex comprises:
i) at least a portion of an MHC molecule sufficient to form a peptide binding groove; and
ii) a candidate antigenic peptide that is covalently linked to the MHC molecule by a peptide linker and which is bound to the peptide binding groove of the MHC molecule, wherein the candidate antigenic peptide is from a library of candidate antigenic peptides, wherein each of the peptides in the library comprises conserved amino acids in a specific sequence sufficient to enable the peptide to bind to the MHC molecule;
b) contacting the baculoviruses or baculovirus-infected cells with a target T cell receptor; and
c) selecting baculoviruses or baculovirus-infected cells that bind to the target T cell receptor.

29. The method of claim 28, further comprising:
d) isolating the selected baculoviruses or baculoviruses from the selected baculovirus-infected cells of step (c);
e) infecting previously uninfected host cells with the isolated baculoviruses of (d) to produce baculoviruses or baculovirus-infected cells enriched for MHC-peptide complexes that bind to the target T cell receptor;
f) contacting the baculoviruses or baculovirus-infected cells from (e) with the target T cell receptor; and
g) selecting baculoviruses or baculovirus-infected cells that bind to the target T cell receptor.

30. The method of claim 29, further comprising isolating the selected baculoviruses or the baculoviruses from the selected baculovirus-infected cells of step (g) and repeating steps (e)-(g) at least one additional time to isolate and identify an MHC-peptide complex that binds to the target T cell receptor.

31. The method of claim 28, wherein the target T cell receptor is labeled with a detectable label.

32. The method of claim 28, wherein the target T cell receptor is expressed on the surface of a cell.

33. The method of claim 28, wherein the target T cell receptor is soluble and immobilized on a substrate.

34. The method of claim 28, wherein the library of candidate antigenic peptides represents from about 10³ to about 10⁹ different candidate antigenic peptides.

35. The method of claim 28, wherein the target T cell receptor is from a patient with a T cell-mediated disease.
